(19) United States
(12) Patent Application Publication
(10) Pub. No.: US 2025/0339038 A1
(43) Pub. Date: Nov. 6, 2025

(54) METHODS AND SYSTEMS FOR PREDICTING THE EFFECT OF INHALED AND INFUSED ANESTHETICS

(71) Applicants: BioVentures, LLC, Little Rock, AR (US); Arkansas Children's Hospital Research Institute, Little Rock, AR (US); Board of Trustees of the University of Arkansas, Little Rock, AR (US)

(72) Inventors: Kevin Sexton, Little Rock, AR (US); Jingxian WU, Little Rock, AR (US); Morten Jensen, Little Rock, AR (US); Hanna Jensen, Little Rock, AR (US); Melvin Dassinger, Little Rock, AR (US); Kaylee Henry, Little Rock, AR (US); Joseph Sanford, Little Rock, AR (US); Ali Al-Alawi, Little Rock, AR (US); Patrick Bonasso, Little Rock, AR (US); Robert Saunders, St. Paul, AR (US)

(21) Appl. No.: 19/034,350

(22) Filed: Jan. 22, 2025

Related U.S. Application Data
(63) Continuation-in-part of application No. 17/919,474, filed on Oct. 17, 2022.
(60) Provisional application No. 63/011,654, filed on Apr. 17, 2020.

(51) Int. Cl. A61B 5/021; A61B 5/00; A61B 5/02

(52) U.S. Cl. CPC A61B 5/02141; A61B 5/02042; A61B 5/7246; A61B 5/7275; A61B 5/7267; A61B 2560/0228; A61B 2560/0257

(57) ABSTRACT

Disclosed herein are devices and systems for analyzing one or more conditions of a patient. The system may comprise a device comprising a tubular body having a first lumen operable to deliver a fluid, at least one sensor at near tip of the tubular body, configured to measure a peripheral venous pressure within a vein of the patient, and at least one processor configured to receive a peripheral venous pressure (PVP) waveform from the at least one sensor, process the PVP waveform, and determine one or more conditions of the patient based on the processed PVP waveform.

### CROSS-REFERENCE TO RELATED APPLICATIONS

This application is a continuation-in-part of U.S. application Ser. No. 17/919,474, filed Oct. 17, 2022, which is a national stage entry of International Application No. PCT/US2021/026977, filed Apr. 13, 2021, which claims priority to U.S. Provisional Application No. 63/011,654, filed Apr. 17, 2020, the contents of which are entirely incorporated by reference herein.

### GOVERNMENT INTEREST STATEMENT

This invention was made with government support under HT9425-24-1-0189 (PR231853) awarded by the Department of Defense, 1U54TR001629-01A1 awarded by the National Institutes of Health, and ECCS1711087 awarded by the National Science Foundation. The government has certain rights in the invention.

### FIELD

The present disclosure relates to systems and methods for predicting the effect of blood volume status and inhaled and infused anesthetics on a patient. More specifically, the disclosure relates to a device that is predicting the volume status and effect of inhaled and infused anesthetics using peripheral venous pressure waveforms.

### BACKGROUND

Existing methods often fail to detect ongoing blood loss until the onset of shock, resulting in poor patient management. Hence, there is an urgent need for a device and/or system that can accurately detect hemorrhage while being part of a comprehensive data collection strategy. To address this, utilizing blood pressure waveforms, a novel method and device of monitoring intravascular volume, is needed to detect volume depletion and hemorrhage at an early stage. This wearable device can effectively detect and monitor internal bleeding or hypovolemic shock, thereby reducing the occurrence of death from bleeding.

A device that consists of a cost-effective wearable equipment for early detection of internal bleeding or hypovolemic shock using veno-arterial crosstalk as an integral component of the predictive algorithm is needed, which in turn will enhance patient care and outcomes in trauma applications by improving the early detection and management of hemorrhage, reducing death, and suffering from bleeding.

The depth of a patient's anesthesia in the hemorrhagic portion of the surgery is controlled by altering the minimum alveolar concentration (MAC) of an inhaled anesthetic, where a higher MAC corresponds to a higher dosage of the anesthetic. The depth in the non-hemorrhagic portion of the surgery is controlled by applying bolus dosages of an infused anesthetic. Anesthetic drugs that patients receive before any intervention change the physiology of the blood circulation in the vessels causing vasodilation to the vessels.

Previous forms of anesthesia depth assessors have been developed for adult patients, but they are not minimally invasive and therefore not appropriate for pediatric patients. Traditional clinical signs such as hypertension, tachycardia and lacrimation are unreliable indicators of depth of anesthesia. Early techniques based on real time signal processing such as the raw or summated EEG, and lower oesophageal contractility, were unreliable. Many methods use a dimensionless monotonic index as a measure of anesthetic depth.

Therefore, there is a need for a minimally invasive method of predicting the effect of inhaled and infused anesthetics, particularly for the pediatric population.

### SUMMARY

This disclosure provides a method of predicting the blood volume and effect of inhaled and infused anesthetics using a device with embedded PVP waveform analysis. The method can utilize a minimally invasive technology, comprising a peripheral intravenous line and a commercial pressure-monitoring transducer, which requires no additional clinical skills.

In an aspect, a method of predicting a hemodynamic state of a patient being administered an anesthetic may include receiving a peripheral venous pressure (PVP) waveform from the patient, cleaning the PVP waveform, transforming the PVP waveform into the frequency domain, and automatically predicting a hemodynamic state of the patient. The prediction may be made using a k-nearest neighbor (k-NN), neural network, random forest, SVM, naïve Bayes, and/or K-means model. The method may further include acquiring the PVP waveform using a peripheral intravenous catheter linked to a pressure transducer and/or measuring the patient's electrocardiogra (ECG) waveform.

Cleaning the PVP waveform may include sectioning the PVP waveform at a pre-selected length of time to create one or more segments, calculating a remainder of the PVP waveform divided by the pre-selected length of time, removing any last points of the PVP waveform that are equal to the PVP waveform remainder, calculating the mean and the standard deviation for each segment, and removing a segment if there is at least one point outside a set number of standard deviations selected by the user.

The hemodynamic state may be a hypervolemic state, an euvolemic state or a hypovolemic state. The anesthetic may be an infused anesthetic, such as propofol, etomidate, benzodiazepines, fentanyl, remifentanil, sufentanyl, morphine, hydromorphone, phenobarbital, pentobarbital, methohexital, ketamine, esketamine, precedex, lidocaine, bupivacaine, ropivacaine, tetracaine, chloroprocaine, clonidine, fentanyl, hydromorphone, morphine, epinephrine, sodium bicarbonate, or glucocorticoids. The patient may be a pediatric patient.

In another aspect, a method of predicting an anesthetic depth of a patient being administered an anesthetic may include receiving a peripheral venous pressure (PVP) waveform from the patient, cleaning the PVP waveform, transforming the PVP waveform into the frequency domain, and automatically predicting the anesthetic depth of the patient. The automatic prediction may be made using a k-nearest neighbor (k-NN), neural network, random forest, SVM, naïve Bayes, and/or K-means model. The method may further include acquiring the PVP waveform using a peripheral intravenous catheter linked to a pressure transducer. The method may also include measuring the patient's ECG and/or determining ECG and PVP waveform coefficients at the heart rate and respiratory rate frequencies.

Cleaning the PVP waveform may include sectioning the PVP waveform at a pre-selected length of time to create one or more segments, calculating a remainder of the PVP waveform divided by the pre-selected length of time, removing any last points of the PVP waveform that are equal to the PVP waveform remainder, calculating the mean and the standard deviation for each segment, and removing a segment if there is at least one point outside a set number of standard deviations selected by the user.

The anesthetic depth may be a minimum alveolar concentration (MAC) dosage. The anesthetic may be an inhaled anesthetic such as isoflurane, sevoflourane, desflurane, halothane, or nitrous oxide. The patient may be a pediatric patient.

Another aspect provided herein is a device having at least one non-transitory computer readable medium storing instructions which when executed by at least one processor, cause the at least one processor to: receive a peripheral venous pressure (PVP) waveform from a patient administered an anesthetic, clean the PVP waveform, transform the PVP waveform into the frequency domain, and automatically predict a hemodynamic state of the patient and/or an anesthetic depth of the patient. The automatic prediction may be made using a k-nearest neighbor (k-NN), neural network, random forest, SVM, naïve Bayes, and/or K-means model. The patient may be a pediatric patient. The hemodynamic state of the patient and/or the anesthetic depth of the patient may be predicted automatically. The device may further include a peripheral intravenous catheter linked to a pressure transducer to acquire the PVP waveform. The hemodynamic state may be a hypervolemic state, an euvolemic state or a hypovolemic state and the anesthetic depth may be a minimum alveolar concentration (MAC) dosage. The anesthetic may be an infused anesthetic such as propofol, etomidate, benzodiazepines, fentanyl, remifentanil, sufentanyl, morphine, hydromorphone, phenobarbital, pentobarbital, methohexital, ketamine, esketamine, precedex, lidocaine, bupivacaine, ropivacaine, tetracaine, chloroprocaine, clonidine, fentanyl, hydromorphone, morphine, epinephrine, sodium bicarbonate, or glucocorticoids or an inhaled anesthetic such as isoflurane, sevoflourane, desflurane, halothane, or nitrous oxide.

Another aspect provided herein is a system for analyzing one or more conditions of a patient. The system can include a device and at least one processor. The device can include a tubular body including a first lumen and at least one sensor near a tip of the tubular body. The first lumen can be operable to deliver a fluid. The at least one sensor can be configured to measure characteristics of a peripheral venous pressure waveform (PVP) within a vein of the patient. The at least one processor can be configured to receive a PVP waveform from the at least one sensor, process the PVP waveform, and determine a hemodynamic state of the patient based on the processed PVP waveform.

In some aspects, processing the PVP waveform can include cleaning the PVP waveform. In some aspects, cleaning the PVP waveform includes sectioning the PVP waveform at a pre-selected length of time to create one or more segments, calculating a remainder of the PVP waveform divided by the pre-selected length, removing any last points of the PVP waveform that are equal to the PVP waveform remainder, calculating the mean and the standard deviation for each segment, and removing a segment if there is at least one point outside a set number of standard deviations selected by the user. In some aspects, determining the hemodynamic state includes transforming the PVP waveform into a frequency domain.

In some aspects, the device is configured to couple to a fluid infusion system. In some aspects, the tubular body has a second lumen. In some aspects, the at least one sensor is disposed within the second lumen. In some aspects, the system further includes a valve in fluid communication with the first lumen. In some aspects, the device further includes a slot in an exterior wall of the tubular body and a cover operable to enclose at least a portion of the slot. In some aspects, the at least one sensor is disposed within the slot. In some aspects, the at least one sensor is operable to receive a set of calibration data.

Another aspect provided herein is a device for analyzing one or more conditions of a patient. The device can include a tubular body and at least one sensor. In some aspects, the tubular body can include a first lumen operable to deliver a fluid. In some aspects, the at least one sensor can be near a tip of the tubular body. In some aspects, the at least one sensor can be configured to measure a peripheral venous pressure within a vein of the patient.

In some aspects, the tubular body can further include a second lumen. In some aspects, the at least one sensor can be disposed in the second lumen. In some aspects, the at least one sensor includes a vented pressure sensor. In some aspects, the at least one sensor is disposed within a slot at an exterior wall of the tubular body. In some aspects, the device can further include a cover. In some aspects, the cover can be operable to enclose one or more extruded wires coupled to the at least one sensor disposed within the slot. In some aspects, the at least one sensor can include an absolute sensor. In some aspects, the at least one sensor can further include a barometric pressure sensor. In some aspects, the at least one sensor can include a wireless transmitter. In some aspects, the at least one sensor can be operable to measure pressure at a frequency greater than a frequency of a peripheral venous pressure. In some aspects, the at least one sensor can be operable to receive a set of calibration data.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIG. 1 is a diagram of the prediction method in one example.

FIG. 2A shows an example euvolemic patient's preoperative peripheral venous pressure (PVP) waveform in time domain.

FIG. 2B shows an example euvolemic patient's intraoperative PVP waveform.

FIG. 2C shows an example euvolemic patient's preoperative frequency domain PVP and piezoelectric waveforms.

FIG. 2D shows an example euvolemic patient's intraoperative frequency domain PVP and piezoelectric waveforms.

FIG. 3A shows an example isoflurane patient's PVP waveform in the time domain for MAC group 1.

FIG. 3B shows an example isoflurane patient's PVP waveform in the time domain for MAC group 2.

FIG. 3C shows an example isoflurane patient's PVP waveform in the frequency domain and EKG waveform for MAC group 1.

FIG. 3D shows an example isoflurane patient's PVP waveform in the frequency domain and EKG waveform for MAC group 2.

FIG. 4 illustrates example system embodiments.

FIG. 5 illustrates an example machine learning environment.

FIG. 6 is an example of movement interfering with collection of the PVP waveform.

FIG. 7 is an example of cleaning the PVP waveform, where the box with the cross encloses an unwanted data section that will be removed.

FIG. 8A is a receiver operating characteristic (ROC) curve plotted as 1−specificity vs sensitivity for propofol.

FIG. 8B is a ROC curve plotted as 1−specificity vs sensitivity for MAC classification.

FIG. 9A is an exemplary porcine CVP waveform before bleeding.

FIG. 9B is an exemplary porcine ECG waveform before bleeding.

FIG. 9C is an exemplary power spectral density of the CVP and ECG with a respiratory rate and a pulse rate.

FIG. 9D is an exemplary correlation coefficient plot.

FIG. 10 is a flow diagram of an exemplary system for volume status assessment using PVP analysis.

FIG. 11 is a flow diagram of an exemplary method for volume status assessment using PVP analysis.

FIGS. 12A-12B show an example combined dehydrated and resuscitated PVP data test waveform in the time domain.

FIGS. 13A-13C show examples of raw resuscitated waveform and sampled resuscitated waveforms with exemplary voltage gains in the time domain.

FIGS. 14A-14C show examples of raw dehydrated waveform and sampled dehydrated waveforms with exemplary voltage gains in the time domain.

FIG. 15A shows a set of exemplary resuscitated PVP spectral density waveforms.

FIG. 15B shows a set of exemplary dehydrated PVP spectral density waveforms.

FIG. 16 shows an example tubular body with a single lumen, comprising a pressure sensor integrated into the tubular body.

FIG. 17 shows an example dual-lumen tubular body comprising a first lumen and a second lumen, and a vented pressure sensor slot integrated into the dual-lumen tubular body.

FIG. 18 shows an example system for monitoring PVP waveforms and delivering fluid to a patient.

FIGS. 19A-19B show example visual representations of a vented pressure sensor and an absolute pressure sensor, respectively.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Provided herein are devices, systems, and methods for detecting and/or collecting peripheral venous pressure (PVP) waveforms of a patient, as well as determining, diagnosing, and/or predicting one or more conditions (e.g., hemodynamic states). For example, the devices may collect PVP waveforms for further analysis. In some examples, the devices and systems can measure PVP waveforms in a patient and use the PVP waveforms to diagnose various hemodynamic states of the patient (e.g., dehydration, effects of anesthesia, blood loss, etc.).

In some examples, the method can predict the blood volume and effect of inhaled and infused anesthetics using a device with embedded PVP waveform analysis.

The devices, systems, and methods utilize a minimally invasive technology, comprising a peripheral intravenous line and a pressure-monitoring transducer. The devices, systems, and methods can be easy to use and require little or no specialized clinical training.

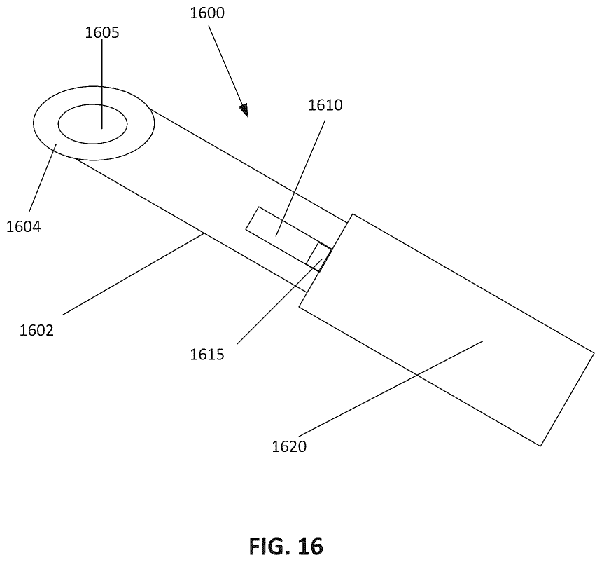

FIG. 16 illustrates a device 1600. The device 1600 can be operable to connect to a fluid conduit of an infusion system (e.g., intravenous (IV) infusion system). In some examples, the device 1600 can be operable to measure PVP waveforms within a patient. In some examples, the device 1600 can be operable to deliver a fluid (e.g., saline, anesthesia, or other liquid medications) to the patient. For example, all, or a portion of, the device 1600 is operable to be inserted into the patient's veins.

In some examples, the device 1600 can be a wearable device. For example, the device 1600 can be worn by a user (e.g., patients, soldiers in the field, etc.). The PVP waveforms measured by the device 1600 can be used to predict blood volume to determine early signs of hemorrhage and/or blood loss before a patient goes into shock.

As illustrated in FIG. 16, the device 1600 can include a body 1602 (e.g., tubular body). The body 1602 can include a coupling mechanism (not shown) operable to connect to an infusion system. The body 1602 can further include a tip 1604. The body 1602 can also include at least one lumen 1605. In some examples, the at least one lumen 1605 can be in fluid communication with an infusion system, such that the at least one lumen 1605 can deliver fluid provided from the infusion system to the patient through the tip 1604. In some examples, the at least one lumen 1605 can be a fluid lumen operable to inject fluid through the body 1602 and into the patient's vein.

The device 1600 can further include at least one sensor 1615 integrated into the body 1602. The at least one sensor 1615 can be operable to enter into the patient's body when the device 1600 is inserted into the patient. In some examples, the at least one sensor 1615 can be disposed (e.g., housed) within a slot 1610 of an exterior wall of the body 1602. In some examples, a cover 1620 can enclose a portion of the slot 1610, such that the at least one sensor 1615 is exposed to the environment within the patient, but the remainder of the slot 1610 is covered. In some examples, the pressure sensor is coupled to one or more wires (not shown) that run along the outside of the body 1602 through the slot. The cover 1620 is affixed or wrapped over and around a portion of the body 1602 a to enclose the one or more wires (e.g., extruded wires). By enclosing the one or more wires, the cover 1620 may prevent any current leakage to the patient and/or the at least one sensor 1615. In some examples, the cover 1620 may also provide additional protection to the at least one sensor 1615, as sealing off the sensor can reduce the risk of fluid ingress and corrosion. In one example, a cover 1620 is a thin heat shrink wrap.

In some examples, the body 1602 can include a needle. For example, the tip 1604 can include a needle edge for ease of insertion into the patient.

In some examples, the at least one sensor 1615 is operable to measure and collect PVP waveforms of the patient. For example, the at least one sensor can be a absolute pressure sensor, a vented pressure sensor, a pressure transducer, or any other sensor operable to measure PVP waveforms. In some examples, an absolute pressure sensor may require a barometric pressure sensor to correct changes in atmospheric pressure readings in order to provide true pressure measurements. In some examples, the barometric pressure sensor can be disposed within a portion of the slot 1610 that extends outside of the patient while the device is inserted in the patient or in an interfacing hardware outside of the patient while the device 1600 is in use. In some examples, the absolute sensor compares the measured pressure to a set atmospheric pressure, however, the pressure in the environment (e.g., external patient environment) can change or not be the same as the set atmospheric pressure. The barometric sensor can measure the atmospheric pressure in the environment (e.g., external patient environment) and the atmospheric pressure reading can be used to accurately adjust the absolute pressure sensor reading (e.g., by using the atmospheric pressure measured by the barometric pressure sensor rather than the preset atmospheric pressure of the absolute pressure sensor).

In some examples, the at least one sensor 1615 can be operable to measure pressure (sample) at a frequency greater than a frequency of peripheral venous pressure change. For example, the at least one sensor 1615 can be operable to measure pressure (sample) at a frequency at least two times the highest frequency of peripheral venous pressure change (the signal).

In some examples, the at least one sensor 1615 includes a wireless transmitter for transmitting data (e.g., measurements) to a computing device and/or processor for data analysis. In some examples, the at least one sensor 1615 can be a wired sensor. The wires can be operable to transmit the data (e.g., measurements) to a computing device and/or processor.

In some examples, the at least one sensor 1615 can be calibrated by a set of calibration data. For example, the calibration data can be received by the sensor and the sensor can be calibrated to measure PVP and/or PVP waveforms in a patient. The calibration data can depend on the type of sensor or sensors. In some examples, calibrating the at least one sensor 1615 also includes zeroing the at least one sensor 1615 at the temperature it will be used at. For example, when the at least one sensor 1615 is to be used in the patient's body to measure PVP, the at least one sensor 1615 can be zeroed at a temperature approximately equal to body temperature (e.g., 37 degrees Celsius).

While the at least one sensor 1615 is described in terms of a pressure sensor, it will be appreciated that the at least one sensor 1615 can be any other kind of sensor configured to measure parameters for a peripheral venous waveform. For example, the at least one sensor 1615 can include one or more flow sensors or other sensors operable to measure parameters for a desired peripheral venous waveform. In some examples, the at least one sensor 1615 can measure other characteristics of a peripheral venous pressure waveform (e.g., amplitude, volume, etc.).

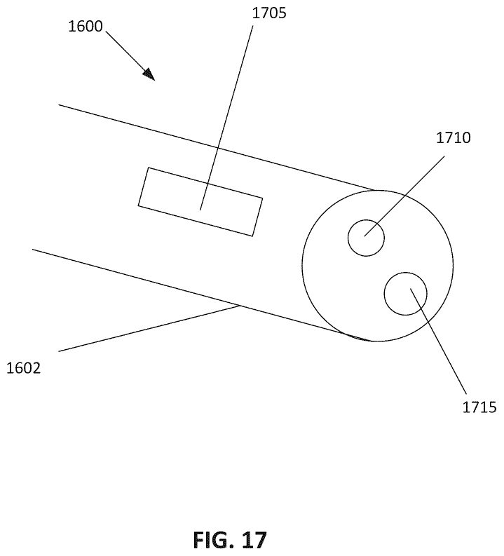

FIG. 17 shows another example of the device 1600. The at least one lumen 1605 can include a first lumen 1710 and a second lumen 1715. In some examples, the at least one lumen 1605 can include two or more lumens. In some examples, the at least one lumen 1605 can include one lumen to ten lumens, or any number of lumens therebetween. In some examples, the at least one lumen 1605 can include more than ten lumens.

In an example, at least one sensor (not shown) can be integrated into one of the first lumen 1710 and the second lumen 1715. In this example, the other lumen without the at least one sensor can deliver fluid, such as intravenous saline solutions, infused anesthetics, or other liquid medications, to the patient, so that the at least one sensor 1615 is fully separated from the fluid being delivered. For example, the first lumen 1710 can be used as a fluid lumen to inject fluid through the body 1602 and into the patient's vein. The second lumen 1715 can be a dedicated lumen that incorporates the at least one sensor 1615 sensor. In some examples, the body 1602 can be a dual-lumen catheter or a dual-lumen cannula. In some examples, the infused anesthetics can include propofol, etomidate, benzodiazepines, fentanyl, remifentanil, sufentanyl, morphine, hydromorphone, phenobarbital, pentobarbital, methohexital, ketamine, esketamine, precedex, lidocaine, bupivacaine, ropivacaine, tetracaine, chloroprocaine, clonidine, fentanyl, hydromorphone, morphine, epinephrine, sodium bicarbonate, or glucocorticoids or an inhaled anesthetic such as isoflurane, sevoflourane, desflurane, halothane, or nitrous oxide.

In some examples, a vented sensor slot 1705 is disposed over one of the first lumen 1710 and the second lumen 1715 in an exterior wall of the body 1602. In some examples, the vented sensor slot 1705 is in communication with the at least one sensor 1615 within the first lumen 1710 or second lumen 1715. In some examples, the vented sensor slot 1705 can be operable to be outside of the patient while the device 1600 is inserted in the patient. While the device 1600 is inserted in the patient, the at least one sensor 1615 (e.g., within the first lumen 1710 or the second lumen 1715) is within the patient, the vented sensor slot 1705, in communication with the first lumen 1710 or second lumen 1715, is outside of the patient (e.g., exposed to the atmospheric pressure of the external patient environment). When the at least one sensor is a vented sensor (e.g., vented pressure sensor), the vented sensor slot 1705 can allow the vented pressure sensor to communicate with atmospheric pressure (e.g., the pressure in the patient environment) when measuring PVP waveforms. In this manner, the vented pressure sensor can be operable to accurately determine the PVP in relation to the atmospheric pressure in the environment (e.g., the atmospheric pressure of the external environment of the patient). In this manner, the vented pressure sensor can self-correct for differences in the atmospheric pressure.

In some examples, the device 1600 operates without delivering a fluid. For example, the device 1600 can record and analyze PVP waveforms to determine blood volume status. The blood volume status can be indicative of hemorrhage and/or blood loss, thereby providing earlier diagnosis before a user goes into shock. The determinations of blood volume status can be highly beneficial when a user is wearing the device 1600 outside of a hospital or medical facility. For example, soldiers in the field can wear the device 1600 and their blood volume status can be monitored, thereby providing early diagnosis of blood loss or hemorrhage before the soldier goes into shock. The device 1600 can effectively detect and monitor internal bleeding or hypovolemic shock, thereby reducing the occurrence of death from bleeding.

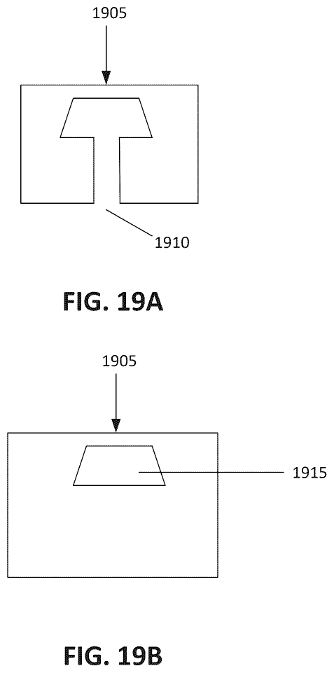

FIG. 19A illustrates a vented pressure sensor. As illustrated, the vented pressure sensor measures a venous pressure 1905 to record a venous pressure (PVP) waveform for frequency response analysis, as described further herein. The vented pressure sensor is open on the back side of a thin diaphragm that deflects with changes in pressure and constantly references atmospheric pressure 1910. The continuous referencing of the atmospheric pressure 1910 can allow the vented pressure sensor to “self-correct” for changes in atmospheric pressure 1910, so the atmospheric pressure 1910 does not need a barometric pressure sensor. In some examples, the vented pressure sensor can be disposed within the first lumen 1710 or second lumen 1715 of the device. In some examples, the vented pressure sensor can be in communication with the vented sensor slot 1705.

FIG. 19B illustrates the absolute pressure sensor. As illustrated, the absolute pressure sensor is fully sealed under the diaphragm to form a vacuum 1915, which therefore provides a pressure reading relative to a constant baseline pressure. In an example, the pressure reading requires a true pressure value, which requires correcting readings from the absolute pressure sensor for changes in atmospheric pressure readings. In this example, the absolute pressure sensor may require a second pressure sensor (e.g., barometric pressure sensor) in order to provide a true pressure measurement. In some examples, the barometric pressure sensor can be placed on or in the body 1602. In other examples, the barometric pressure sensor can be placed in an interfacing hardware. In some examples, the barometric pressure sensor can measure an atmospheric pressure, which can be provided to calculate the true pressure reading by making the atmospheric pressure a reference point, thereby replacing an absolute zero reference point of the absolute pressure sensor. In some examples, the absolute pressure sensor can be disposed within the slot 1610 (or vented sensor slot 1705) of the device 1600.

In some examples, the device 1600 can include a valve within the body 1602 and in fluid communication with the fluid delivery lumen of the at least one lumen 1605. The valve can have an open state and a closed state. In the open state, fluid can be delivered to the patient through the first lumen. In the closed state, the valve can prevent fluid from flowing past the valve in the first lumen, thereby preventing fluid from being delivered to the patient. By preventing fluid from being delivered to the patient, the valve can allow the at least one sensor to measure pressure (e.g., PVP waveforms) more accurately, by preventing additional noise caused by fluid flowing through the first lumen and into the vein, blood vessel, artery, etc. In some examples, the valve can be a mechanically actuated valve. In some examples, the valve can be an electronically actuated valve. The electronically actuated valve can be actuated between the open state and the closed state by the at least one processor (e.g., via a user input). When the valve is in the closed state, accurate PVP waveforms can be measured by the at least one sensor 1615.

In some examples, integrating the at least one sensor 1615 into the slot 1610 of the body 1602 or within the first lumen 1710 or second lumen 1715 reduces and/or prevents the at least one sensor 1615 from being bent as the device 1600 is inserted into the patient. For example, the at least one sensor 1615 is mechanically supported by the rigidity of the body 1602, thereby preventing and/or reducing any risk of the at least one sensor 1615 bending.

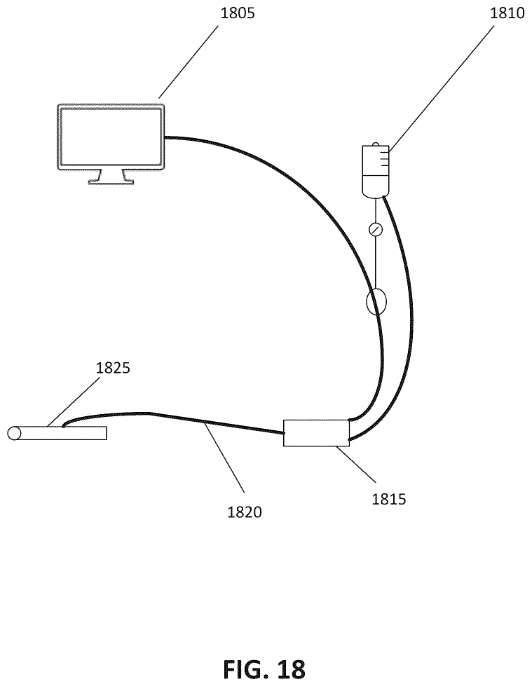

FIG. 18 shows an example system including a computing system 1805, an infusion system 1810, at least one sensor 1815 (e.g., at least one sensor 1615, pressure sensor, pressure transducer, etc.), a delivery tubing 1820, and a cannula 1825. In some examples, the at least one sensor 1815, delivery tubing 1820, and cannula 1825 can be the device 1600 described herein. The computing system 1805 can include any of the computing systems described herein. The computing system 1805 can be operable to perform any of the methods described herein. The computing system 1805 can include any of the processors, displays, or other computing components described herein.

Provided herein are methods of predicting the effect of anesthetics and/or other medical fluids on a patient using peripheral venous pressure (PVP) waveforms. The methods of predicting the effect of anesthetics on a patient may be used to prevent overdosage or underdosage of anesthesia during a pediatric medical operation. In some examples, infused and inhaled anesthetics may have an impact on the PVP waveforms and machine learning may be used to automatically identify how anesthetics are affecting a patient by analyzing the patient's PVP waveforms. The method may be nearly instantaneous, minimally invasive, work with both infused and inhaled anesthetics, and be applicable to pediatric populations. In some examples, the prediction can also include a diagnosis or determination of one or more hemodynamic conditions of the patient.

Analysis of peripheral venous pressure (PVP) waveforms is a novel method of monitoring intravascular volume, especially in cases of dehydration and hemorrhage. PVP has been shown to be a predictor of dehydration in pediatric patients. However, PVP waveforms can potentially be confounded by parameters other than volume status, such as anesthetic agents, while collecting the data. Anesthetic drugs, inhaled or infused, influence the PVP signal significantly.

The methods provided herein determined a significant relationship between both infused and inhaled anesthetics and the PVP waveform, as the PVP signal is influenced by the different hemodynamics states of the body.

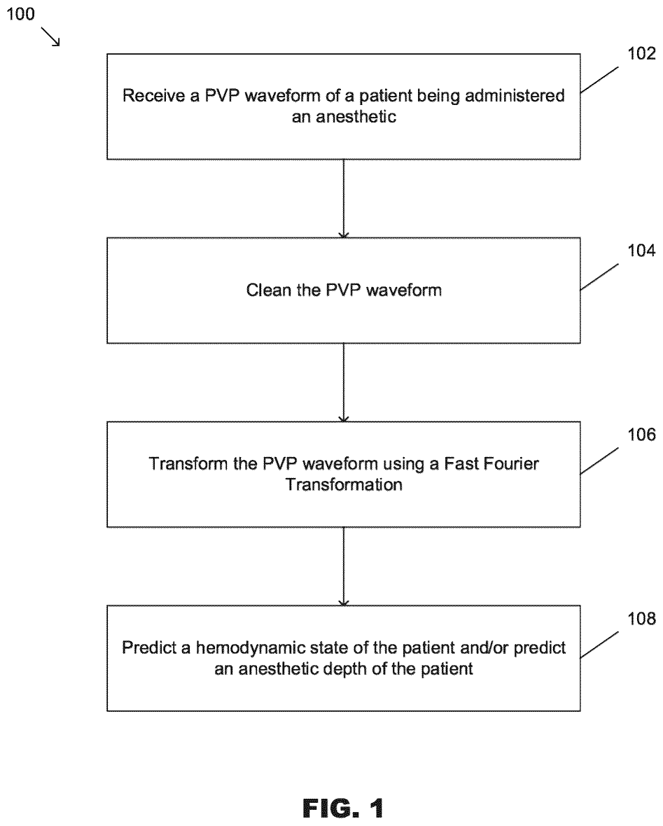

The overall framework of the prediction method 100 is shown in FIG. 1. At step 102, the prediction method 100 may include receiving a peripheral venous pressure (PVP) waveform from a patient being administered an anesthetic. In at least one example, the patient is a pediatric patient. In additional examples, the pediatric patient may be an infant. The anesthetic may be an infused anesthetic or an inhaled anesthetic. Non-limiting examples of inhaled anesthetics include isoflurane, sevoflurane, desflurane, halothane, and nitrous oxide. Isoflurane causes vasodilation in the peripheral blood vessels and alters the blood flow. The infused anesthetic may be an infused gamma-aminobutyric acid (GABA) agonist anesthetic, an infused narcotic, an infused barbiturate, an infused NMDA antagonist, an infused alpha agonist, or an infused neuraxial anesthetic. Non-limiting examples of GABA agonists include propofol, etomidate, and benzodiazepines. Propofol is an anesthetic drug that causes immediate vasodilation and relaxes the patient's vessels, which decreases the pressure in the vessels. Non-limiting examples of infused narcotics include fentanyl, remifentanil, sufentanyl, morphine, and hydromorphone. Non-limiting examples of infused barbiturates include phenobarbital, pentobarbital, and methohexital. Non-limiting examples of infused NMDA antagonists include ketamine and esketamine. Non-limiting examples of infused alpha agonists include precedex. Non-limiting examples of neuraxial anesthetics include lidocaine, bupivacaine, ropivacaine, tetracaine, chloroprocaine, clonidine, fentanyl, hydromorphone, morphine, epinephrine, sodium bicarbonate, and glucocorticoids.

In various examples, a device may include an apparatus for acquiring the PVP waveform and at least one processor for performing the steps of the method 100. The device may continuously measure the PVP waveform and predict the anesthetic depth in the patient before and during a medical operation. In some examples, the PVP waveform may be acquired using a peripheral intravenous catheter linked to a pressure transducer. PVP can be measured via a peripheral IV, making it easy to access and measure compared to central venous pressure (CVP). In at least some examples, the PVP waveform may be measured via a peripheral IV in the arms or legs of the patient or at any location on the patient that may receive a peripheral IV. CVP is traditionally used in assessing the overall circulatory status of a patient in an intensive care or operative setting, and to guide resuscitation. Several studies have shown that CVP and PVP correlate significantly. However, use of PVP waveforms is a less invasive method of measuring volume status. The PVP waveform may be acquired by any method known in the art. In some examples, the PVP waveform may be acquired through a piezoelectric crystal. In additional examples, the PVP waveform may be acquired transcutaneously.

At step 104, the method 100 may include cleaning the PVP waveform. Cleaning the PVP waveform may remove unwanted motion artifacts. In various examples, the PVP waveform may be cleaned automatically. Cleaning the PVP waveform automatically may include sectioning the PVP waveform at a pre-selected length of time to create one or more segments, calculating a remainder of the PVP waveform divided by the pre-selected length of time, removing any last points of the PVP waveform that are equal to the PVP waveform remainder, calculating the mean and the standard deviation for each segment, and removing a segment if there is at least one point outside a set number of standard deviations selected by the user.

At step 106, the method 100 may include transforming the PVP waveform into the frequency domain. In some examples, the PVP waveform may be transformed using a Fast Fourier Transformation (FFT). The venous system is highly compliant and can accommodate large changes in volume with minimal changes in pressure. However, the detection of the subtle changes in PVP waveforms as a result of volume loss is made possible due to signal amplifying technologies that can extract hemodynamic signals in the frequency domain by using FFT. The frequency domain PVP signals may then be analyzed with advanced statistical and machine learning algorithms. Venous waves are generated by the cardiac cycle and propagated as harmonics. The f1 waveform which correlates with the heart rate, has been shown to be affected already by very mild hypovolemia. The FFT of a PVP waveform correlates with volume status more sensitively than standard vital signs monitoring. However, despite the robust evidence of the correlation between PVP waveforms and volume status, both the exact mechanism behind this link, and potential confounding parameters have not been thoroughly investigated.

At step 108, the method 100 may include automatically predicting a hemodynamic state of the patient and/or automatically predicting an anesthetic depth of the patient. In some examples, the method may automatically predict a hemodynamic state and/or automatically predict an anesthetic depth using a k-nearest neighbor (k-NN), neural network, random forest, SVM, naïve Bayes, and/or K-means model. In some examples, the prediction of the hemodynamic state or the anesthetic depth may prevent overdosage or underdosage of anesthesia during a medical operation, in particular a pediatric medical operation. Predicting the hemodynamic state of the patient or predicting the anesthetic depth may be done automatically. In at least some examples, the prediction may be performed in real-time (i.e. instantaneous/immediate), or have a delay of up to 5 seconds, up to 10 seconds, up to 30 seconds, or up to 1 minute from the time the PVP waveform is received.

In some examples, the hemodynamic state predicted may be a hypervolemic state, an euvolemic state, or a hypovolemic state. For example, the hemodynamic state of a patient can include hydration levels (e.g., hydrated or dehydrated), a anesthetics dosage level, sepsis, fluid responsiveness, or vasoactive medication responsiveness, or a combination thereof. In some examples, the method may predict if the patient is dehydrated or hydrated at the time of signal collection. This prediction may be useful to a physician because if a patient is dehydrated, then their veins are more constricted than if they were hydrated. The PVP waveforms may be altered by hemodynamic state as well as anesthesia.

The depth of the patient's anesthesia both in a hemorrhagic and non-hemorrhagic portion of surgery is controlled by altering the minimum alveolar concentration (MAC) of the anesthetic. The depth of a patient's anesthesia in the hemorrhagic portion of the surgery may be controlled by altering the MAC of an inhaled anesthetic. The depth in the non-hemorrhagic portion of the surgery may be controlled by applying bolus dosages of an infused anesthetic. In some examples, predicting the anesthetic depth of the patient may include predicting the patient's MAC dosage or MAC group. In various examples, the MAC dosage may be a MAC group of 1, 2, 3, 4, 5, or 6, where a higher MAC corresponds to a higher dosage of anesthetic. Predicting the MAC allows for an anesthesiologist to verify that the MAC dosage they've applied has changed the waveform exactly as intended. Also, the anesthetic depth may be assessed by the MAC that is predicted. For example, if the MAC group predicted is 3 or higher, then it is known that the patient has a high anesthetic depth. For patients receiving an infused anesthetic, the method may determine if the anesthetic is still making an effect on the waveform (e.g. 0 (no presence) or 1 (presence)).

In some examples, the prediction method may predict preoperative (i.e. the absence of anesthesia) and intraoperative signals (i.e. the presence of anesthesia) and/or may classify an arbitrary PVP signal to its correct MAC dosage or infused anesthetic bolus presence. Being able to see a significant difference in the PVP signal at different hemodynamic states has an important impact to the medical field. First, it helps the physicians to make an immediate decision in emergency situations. Also, showing a significant relationship between the anesthetic drugs, inhaled and infused, and the PVP implies that the consequent changes in vascular resistance due to the anesthetic drugs are reflected in the vein circulation and in the peripheral veins. The prediction methods herein may accurately estimate the volume status of a patient to guide triage and remediation. This may be a significant enhancement in various care settings, including but not limited to surgery, pediatrics, and military use.

The prediction method may utilize a prediction model such as a k-nearest neighbor (k-NN), neural network, random forest, SVM, naïve Bayes, and/or K-means model to predict the hemodynamic status or the anesthetic depth. The prediction model may be previously trained with anesthetic dosages and know how many separate groups of anesthetic dosages are available. Therefore, the prediction model may predict the anesthetic dosage or presence at each time point by comparing the cleaned and transformed PVP waveform to known waveforms (that were used to train the algorithm) in each anesthetic group to see which is most similar.

In some examples, the prediction method may correctly predict at least 77% of euvolemic and hypovolemic groups. The k-NN models of the anesthetic drugs may be able to correctly predict correctly at least 85% of the preoperative and intraoperative signals of the pyloric stenosis patients and the different isoflurane dosages of the craniosynostosis patients.

In some examples, the PVP waveform can capture direct responses to a micro-stimulus, such as micro-dose (e.g., small fluid bolus) of infused fluid medications. In some examples, the micro-dose can include medications besides infused medications, such as inhaled medications, other types of fluids or gas treatments. The micro-stimulus can trigger signal changes that are captured and reflected in the waveform. In some examples, the responses can be monitored continuously over the course of continuous microdosing of fluids. The responses can be indicative of an efficacy of a certain treatment. In this manner, a medical professional can determine whether a certain treatment (e.g., fluid, infused medication, inhaled medication, etc.) will have the desired effect on the patient.

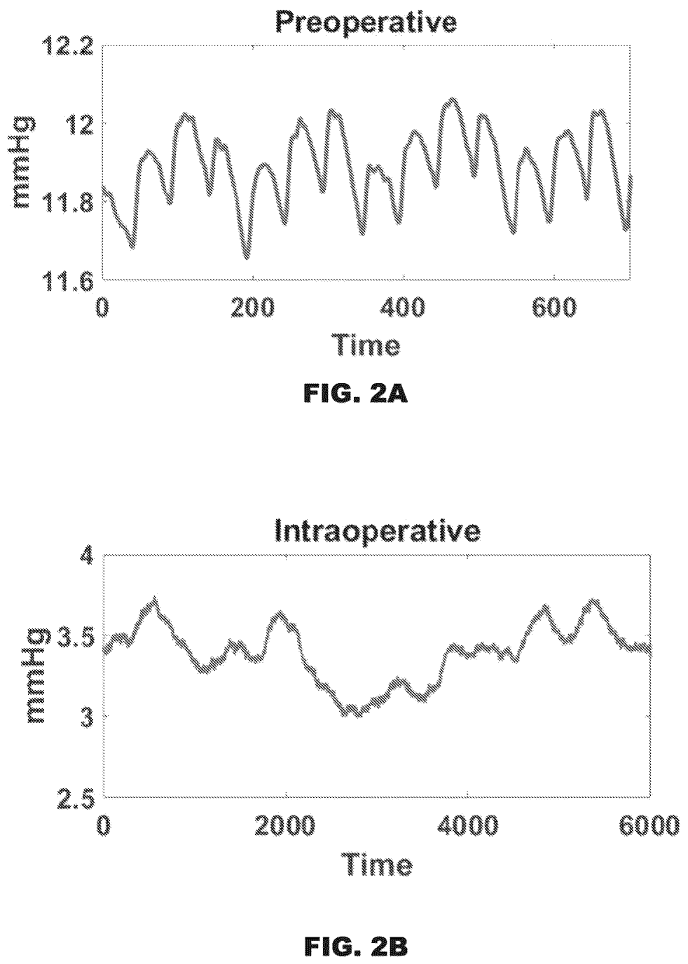

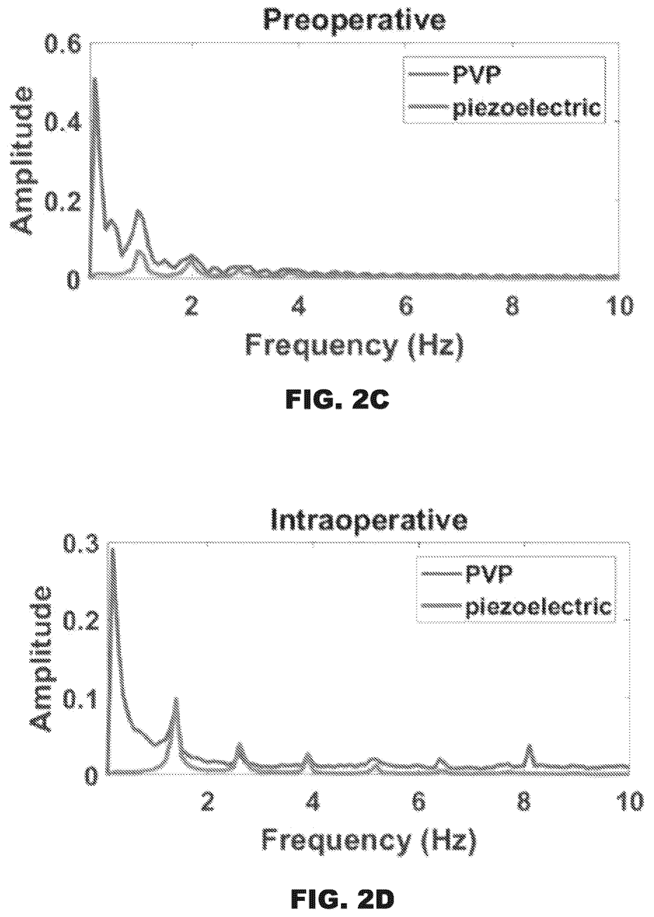

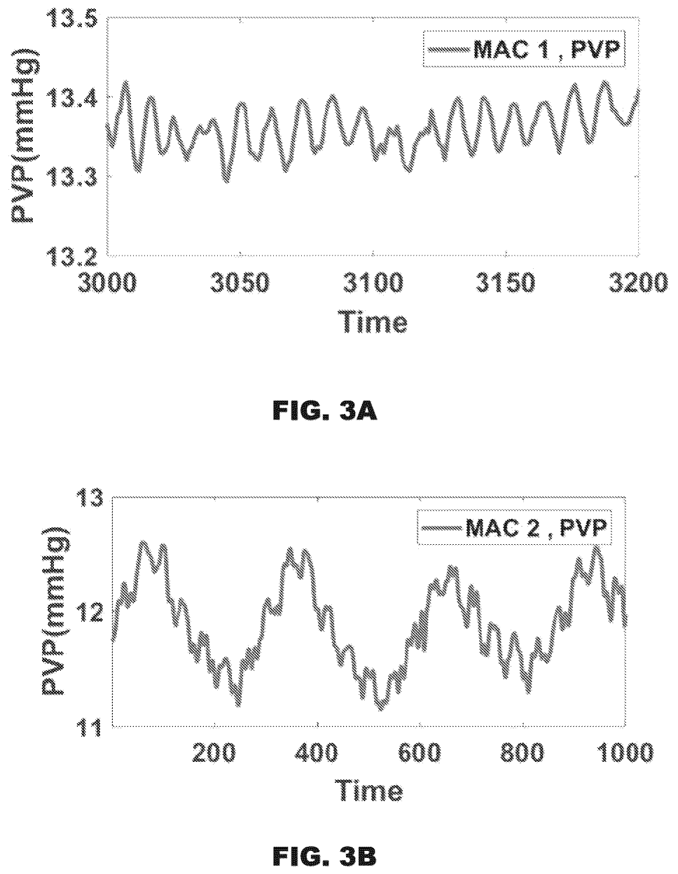

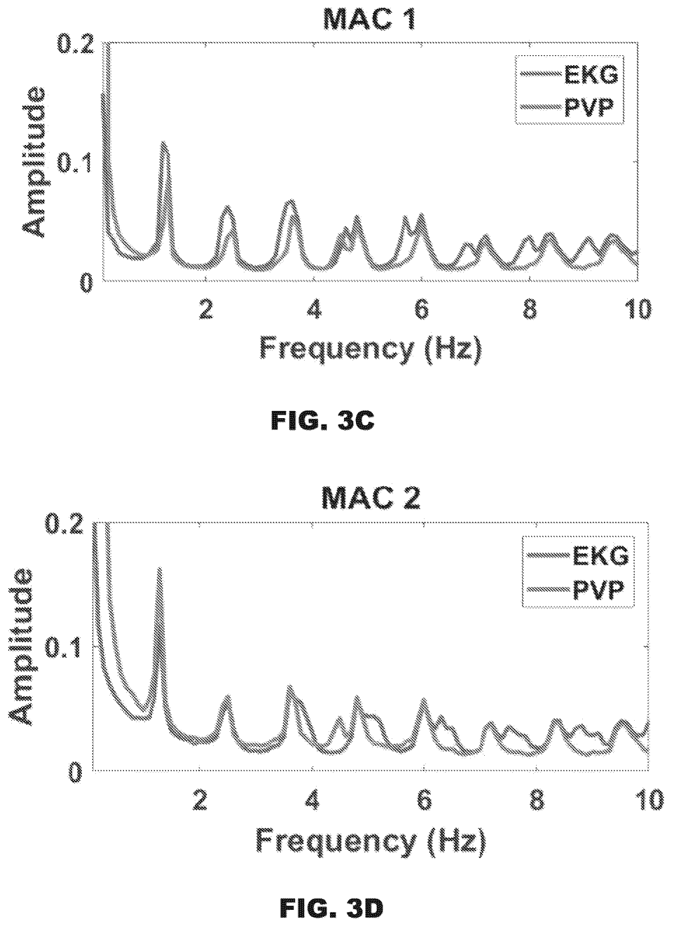

In one example, when propofol is administered, the PVP amplitude of the intraoperative waveform decreases compared to the amplitude of the preoperative waveform. The relationship between propofol and PVP is illustrated in FIGS. 2A-2B, where the PVP amplitude in time domain is lower when propofol was introduced and the PVP harmonics follow the piezoelectric. After administering an infused anesthetic, the PVP amplitude directly decreases. The piezoelectric and PVP frequencies correlate, showing that pulse rate decreases when the patient is under anesthetics. For the isoflurane patients, whenever MAC increases, the PVP waveform decreases. This demonstrates that increasing MAC immediately dilates the veins and reduces venous pressure; the relationship is illustrated in FIGS. 3A-3B. FIGS. 3A-3D show the PVP amplitude in time domain is lower in higher MAC dosages and the PVP harmonics follow the patient's electrocardiogramaCG/EKG).

In additional examples, the method may further include measuring the patient's ECG. The method may also include determining ECG and PVP waveform coefficients at the heart rate and respiratory rate frequencies. Measuring the ECG along with the PVP may identify the frequency that corresponds to the heart rate and whether it is matching the frequency at the highest peak of the PVP waveform. There is a robust mimicking between the frequency of PVP and the frequency of ECG and the frequencies at the highest amplitude in FIGS. 2C-2D are equal, 1.2 Hz. In human arms and legs, peripheral arteries and veins run in close anatomical proximity, and it is feasible to assume that the pressure in one vessel can carry over to the other. Without being limited to any particular theory, it appears that in hydrated patients, the cross-talk between arteries and veins in direct physical interaction with each other accounts for the signal waveform in frequencies corresponding to heart rate. When the patient has adequate blood volume, the arterial pulse pressure waveform crosses over to the venous side. In dehydrated patients, as the diameter of arteries and veins decreases, the cross-talk is lost and the signal waveform is affected at the frequency of the heart rate. Therefore, the methods herein may take into account the heart rate of a patient, to prevent the limitation of PVP signal analysis.

In some examples, the method may further include preventing overdosage or underdosage of anesthesia during a medical operation. In at least one example, the medical may be a pediatric medical operation. The automatic prediction of the hemodynamic status or anesthetic depth in the patient may inform a physician of how adjust or correct the dosage of anesthesia being administered to the patient to prevent overdosage or underdosage. For example, a minimum and/or maximum anesthetic depth may be provided by the physician or may be pre-set. Then, the dosage administered to the patient may be adjusted to maintain the predicted anesthetic depth within the minimum and maximum values to prevent overdosage or underdosage. The dosage being administered to the patient may be adjusted automatically or may be adjusted manually by the physician.

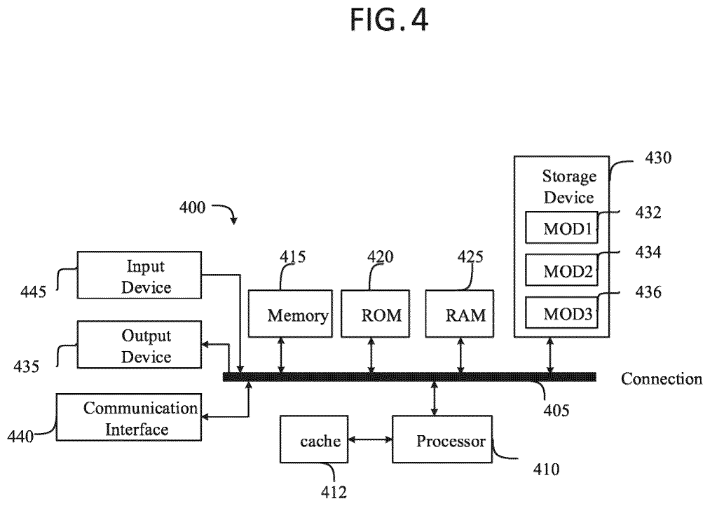

The disclosure now turns to the example system illustrated in FIG. 4 which may be used to implement the methods for predicting a hemodynamic state and/or anesthetic depth of a patient. In an example, a device may include a computing system having at least one processor for predicting a patient's hemodynamic status and/or anesthetic depth. FIG. 4 shows an example of computing system 400 in which the components of the system are in communication with each other using connection 405. Connection 405 can be a physical connection via a bus, or a direct connection into processor 410, such as in a chipset or system-on-chip architecture. Connection 405 can also be a virtual connection, networked connection, or logical connection.

In some examples computing system 400 is a distributed system in which the functions described in this disclosure can be distributed within a datacenter, multiple datacenters, a peer network, throughout layers of a fog network, etc. In some examples, one or more of the described system components represents many such components each performing some or all of the function for which the component is described. In some examples, the components can be physical or virtual devices.

Example system 400 includes at least one processing unit (CPU or processor) 410 and connection 405 that couples various system components including system memory 415, read only memory (ROM) 420 or random access memory (RAM) 425 to processor 410. Computing system 400 can include a cache of high-speed memory 412 connected directly with, in close proximity to, or integrated as part of processor 410.

Processor 410 can include any general purpose processor and a hardware service or software service, such as services 432, 434, and 436 stored in storage device 430, configured to control processor 410 as well as a special-purpose processor where software instructions are incorporated into the actual processor design. Processor 410 may essentially be a completely self-contained computing system, containing multiple cores or processors, a bus, memory controller, cache, etc. A multi-core processor may be symmetric or asymmetric.

To enable user interaction, computing system 400 includes an input device 445, which can represent any number of input mechanisms, such as a microphone for speech, a touch-sensitive screen for gesture or graphical input, keyboard, mouse, motion input, speech, etc. Computing system 400 can also include output device 435, which can be one or more of a number of output mechanisms known to those of skill in the art. In some instances, multimodal systems can enable a user to provide multiple types of input/output to communicate with computing system 400. Computing system 400 can include communications interface 440, which can generally govern and manage the user input and system output, and also connect computing system 400 to other nodes in a network. There is no restriction on operating on any particular hardware arrangement and therefore the basic features here may easily be substituted for improved hardware or firmware arrangements as they are developed.

Storage device 430 can be a non-volatile memory device and can be a hard disk or other types of computer readable media which can store data that are accessible by a computer, such as magnetic cassettes, flash memory cards, solid state memory devices, digital versatile disks, cartridges, battery backed random access memories (RAMs), read only memory (ROM), and/or some combination of these devices.

The storage device 430 can include software services, servers, services, etc., that when the code that defines such software is executed by the processor 410, it causes the system to perform a function. In some examples, a hardware service that performs a particular function can include the software component stored in a computer-readable medium in connection with the necessary hardware components, such as processor 410, connection 405, output device 435, etc., to carry out the function.

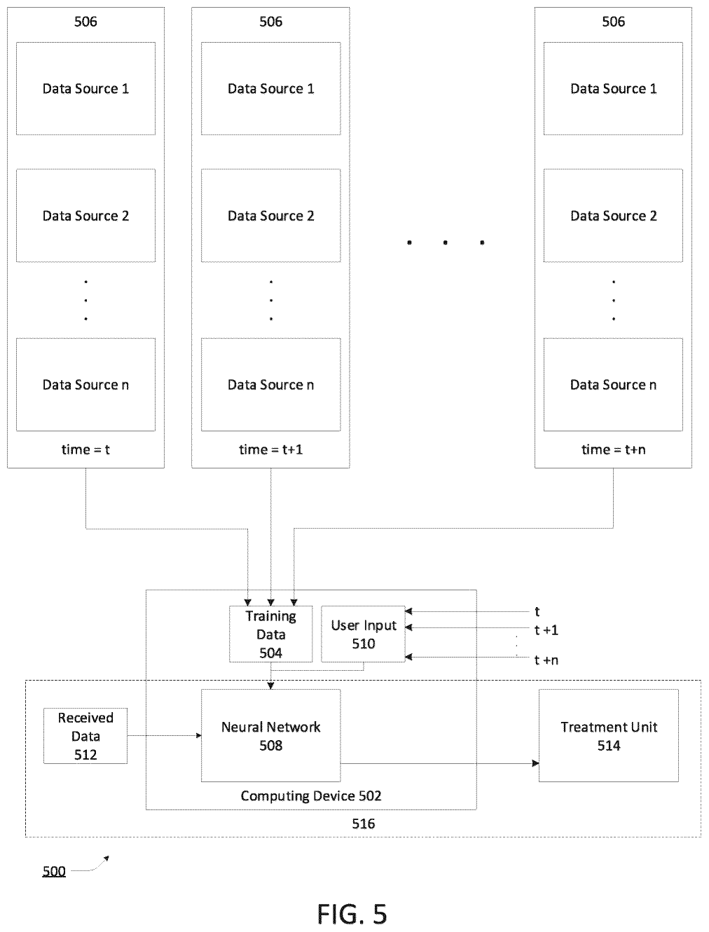

The disclosure now turns to FIG. 5, which illustrates an example machine learning environment 500. The machine learning environment can be implemented on one or more computing devices 502A-N (e.g., cloud computing servers, virtual services, distributed computing, one or more servers, etc.). The computing device(s) 502 can include training data 504 (e.g., one or more databases or data storage device, including cloud-based storage, storage networks, local storage, etc.). In some examples, the training data may include data from patients that have undergone a pyloromyotomy or craniosynostosis surgery with an infused or inhaled anesthetic. The training data 504 of the computing device 502 can be populated by one or more data sources 506 (e.g., data source 1, data source 2, data source n, etc.) over a period of time (e.g., t, t+1, t+n, etc.). In some examples, training data 504 can be labeled data (e.g., one or more tags associated with the data). For example, training data can be one or more PVP waveforms and a label (e.g., MAC value, hemodynamic status, etc.) can be associated with each waveform. The computing device(s) 502 can continue to receive data from the one or more data sources 506 until the neural network 508 (e.g., convolution neural networks, deep convolution neural networks, artificial neural networks, learning algorithms, etc.) of the computing device(s) 502 are trained (e.g., have had sufficient unbiased data to respond to new incoming data requests and provided an autonomous or near autonomous image classification). In some examples, the neural network can be a convolutional neural network, for example, utilizing five layer blocks, including convolutional blocks, convolutional layers, and fully connected layers. In some examples, the neural network may utilize a k-nearest neighbor, neural network, random forest, SVM, naïve Bayes, and/or K-means model. While example neural networks are realized, neural network 508 can be one or more neural networks of various types are not specifically limited to a single type of neural network or learning algorithm.

In other examples, a feature selection can be generated (e.g., group correlated features such that one feature is used for each group). In these instances, cleaned and transformed segments of a PVP waveform are used in a prediction model. The training data can require a minimum or an equivalent number of PVP waveform segments per patient.

In some examples, while not shown here, the training data 504 can be checked for biases, for example, by checking the data source 506 (and corresponding user input) verse previously known unbiased data. Other techniques for checking data biases are also realized. The data sources can be any of the sources of data for providing the PVP waveforms (e.g., IV pressure transducer, etc.) as described above in this disclosure.

The computing device(s) 502 can receive user (e.g., physician) input 510 related to the data source. The user input 510 and the data source 506 can be temporally related (e.g., by time t, t+1, t+n, etc.). That is, the user input 510 and the data sources 506 can be synchronous in that the user input 510 corresponds and supplements the data source 506 in a manner of supervised or reinforced learning. For example, a data source 506 can provide a PVP waveform at time t and corresponding user input 510 can be input of hemodynamic status or MAC group of that PVP waveform at time t. While, time t may actually be different in real-world time, they are synchronized in time with respect to the data provided to the training data.

The training data 504 can be used to train a neural network 508 or learning algorithms (e.g., convolutional neural network, artificial neural network, etc.). The neural network 508 can be trained, over a period of time, to automatically (e.g., autonomously) determine what the user input 510 would be, based only on received data 512 (e.g., PVP waveform, etc.). For example, by receiving a plurality of unbiased data and/or corresponding user input for a long enough period of time, the neural network will then be able to determine what the user input would be when provided with only the data. For example, a trained neural network 508 will be able to receive a PVP waveform (e.g., 512) and based on the PVP waveform determine the hemodynamic status or anesthetic depth that a physician would manually identify (and that would have been provided as user input 510 during training). In some examples, this can be based on labels associated with the data as described above. The output from the trained neural network can be provided to a prediction model 514 for treating a patient. In some examples, the output from the trained neural network can be inputted directly into a prediction model to predict a hemodynamic status and/or anesthetic depth in the patient.

Trained neural network system 516 can include a trained neural network 508, received data 512, and prediction model 514. The received data 512 can be information related to a patient, as previously described above. The received data 512 can be used as input to trained neural network 508. Trained neural network 508 can then, based on the received data 512, label the received data and/or determine a recommended course of action for treating the patient, based on how the neural network was trained (as described above). The recommended course of action or output of trained neural network 508 can be used as an input into the prediction model 514 (e.g., to predict the hemodynamic status and/or anesthetic depth for the patient to which the received data 512 corresponds). In other instances, the output from the trained neural network can be provided in a human readable form, for example, to be reviewed by a physician to determine a course of action.

For clarity of explanation, in some instances the present technology may be presented as including individual functional blocks including functional blocks comprising devices, device components, steps or routines in a method embodied in software, or combinations of hardware and software.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

### EXAMPLES

### Example 1: Acquiring PVP

The impact of anesthetics on PVP waveforms was tested in two anesthetized patient cohorts. The first cohort represented a dehydration setting in infants operated on for pyloric stenosis diagnosed by ultrasound who had been projectile vomiting and admitted prior to undergoing a pyloromyotomy operation during which propofol was infused as an anesthetic. Data was collected after being resuscitated to near euvolemia at the time of operation. The second cohort represented a hemorrhagic setting in infants operated on during a reconstructive, elective craniosynostosis operation.

Due to the vast blood supply to the skull, intra-operative estimated blood loss of 60-70 cc/kg and occasionally up to half of blood volume may need to be replaced utilizing a combination of intravenous fluids (IVF), blood products, and occasionally pressors.

These two cohorts were utilized to determine if anesthetics such as propofol or isoflurane influenced the PVP waveform. After determining the relationship, two machine learning systems were built using a k-nearest neighbor statistical model to predict hydration levels for arbitrary pyloric stenosis PVP waveforms, and also predict MAC for an arbitrary craniosynostosis PVP waveform.

PVP waveforms were collected from 39 pyloric stenosis patients and 9 craniosynostosis patients. For the pyloric stenosis patients, three patients were removed because a Nexiva catheter was used instead of the PIV catheter, resulting in a distinctly different PVP waveform. Two other patients were discarded because their PIV catheters were inserted into the foot. Eleven patients were excluded due to either a flat PVP waveform due to incorrect zeroing of catheter or other circumstances that rendered the data unusable. This resulted in a total of twenty-three patients used for waveform analysis. The patients were further sorted based on their hydrations status when they arrived at the emergency room, either hypovolemic with severe fluid loss, or euvolemic with normal fluid volume. Statistical testing for hypovolemic patients and euvolemic patients were conducted separately. For the isoflurane testing, nine patients were initially included in the study. Two patients were removed because the time of the operation start was not noted when LabChart started recording the PVP, making it difficult to relate MAC and PVP. The seven isoflurane patients were further sorted, based on the number of MAC groups used during the operation. For each patient, there were n MAC groups that were assigned a group number n>0 when MAC fell between n−1 and n−0.1. For example, if MAC ranged between [0-0.9], then it would be classified as MAC group 1.

The average weight of the fifteen enrolled euvolemic pyloric stenosis pediatric patients was 4.14 kilograms (kg) with a standard deviation of 0.68 kg. The average weight of the eight hypovolemic patients was 3.70 kg with a standard deviation of 0.74 kg, which was lower than the euvolemic patients. After enrollment, fluids were given to the hypovolemic patients so that at the time of the operation, the twenty-three patients were all considered euvolemic. The average weight of the enrolled craniosynostosis pediatric patients was 10 kg with a standard deviation of 3.66 kg.

For the pyloric stenosis patients, data points were collected over the entire operation, and for the craniosynostosis patients, data points were collected from the first instance of isoflurane throughout the procedure until isoflurane administration was ceased. PVP waveforms were measured with a 24-gauge Insyte-N Autoguard peripheral intravenous (PIV) catheter. The PIV catheter was connected to a Deltran II pressure transducer using 48-inch arterial pressure tubing. Then, a Powerlab data acquisition system (ADInstruments) was used to connect the hardware setup with LabChart 8 (ADInstruments) to record the waveforms.

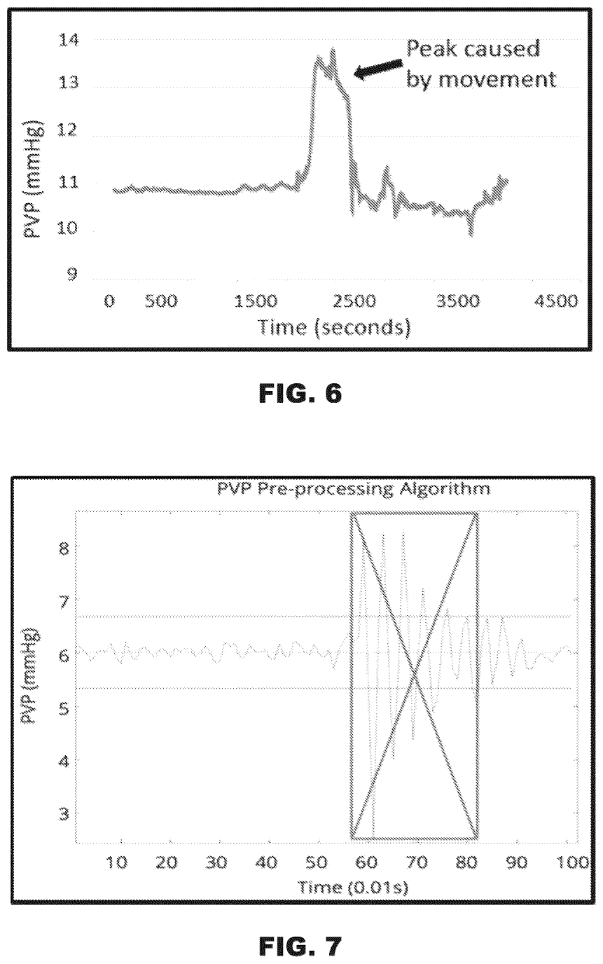

The Deltran pressure transducer detects small movements of the infant, bed movement, infant's crying, or apparatus errors which interferes with the PVP recording. Movement causes large spikes in the recorded waveform as shown in FIG. 6. Other external factors can potentially interfere with the PVP measuring accuracy, such as adjusting the tubing or accidentally hitting the operative table.

### Example 2: Data Cleaning Algorithm and Fast Fourier Transform

Due to waveform contamination due to undesired artifacts mentioned in Example 1, an algorithm was developed using MATLAB to pre-process the data and remove the unwanted sections of the waveforms.

First, the entire PVP waveform was sampled at a rate of 100 Hz from LabChart 8 for each patient. After sampling the waveform, the PVP data was exported into a custom algorithm. For isoflurane patients, the corresponding MAC values were exported alongside the corresponding PVP waveforms. The algorithm takes sections of the PVP data at a user-selected length of time to analyze. The algorithm calculates the remainder of the PVP signal divided by pre-selected time length, the length of the segment, and then remove the last points of the signal that are equal to the PVP signal remainder. These two steps assure that every single segment has the same duration for all the patients. For every section of the PVP waveform signal, the mean value of the data values in that section was calculated, and if any data points in that time section exceeds above or below the user-defined number of standard deviations, then the entire section of data is removed; this method is illustrated in FIG. 7. The algorithm goes through the entire PVP waveform, which can be up to 4 hours long for the isoflurane patients, and removes sections of the data that contain spikes within the segments due to movement. The process takes a maximum of two minutes.

### Example 3: Fast Fourier Transform

Each segment of the PVP signal was transformed into the frequency domain using a Fast Fourier Transform (FFT) function. The analyses were in the frequency domain because it reduces the cost and time of the testing and it is more stable because of the absence of the negative feedback. Also, frequency domain is used to check the dominant amplitudes that reflects many factors such as the heart pulse and respiratory rate.

After the cleaning algorithm, the data was divided into 10-second windows. Each window contains only a continuous waveform, that is, if a section of the waveform was removed during the cleaning process, the waveforms before and after the removed section will not be in the same window. Thus, the frequency domain resolution was 0.1 Hz which represents the distance between two frequency samples. With a time domain sampling rate of 100 Hz, the signal covered a frequency range of 50 Hz. However, only signals from 0 to 20 Hz were used for further processing. When converting the data to the frequency domain, the result is two mirrored values at different frequencies, so using the first 20 Hz ensures that the used bins do not belong to the same frequencies. Furthermore, there is no useful information after the 20th bins since no one can have a heart rate that is greater than 20 Hz. Thus, the total number of bins was 200 and each bin was a feature of the PVP signal at different frequency with 0.1 step frequency size. However, the 200 features were down sampled by a factor of 4 leading to have a 0.4 step frequency size with 50 points for each 10-second segment. The down sampling ensures that the number of observations is more than the number of variables to get reliable results because having 200 frequency features may not be fulfilled in some recorded PVP waveforms due to the small number of observations, less than 200.

### Example 4: Statistical Analysis

During the pyloromyotomy surgery, the patients received propofol. In order to test if the propofol influences the PVP, the intraoperative PVP signal was tested against the preoperative PVP signal when the patient had not received any propofol. It was tested was if the intraoperative and the preoperative PVP waveforms were significantly different; MANOVA was used to test the hypothesis.

The data presented for the isoflurane patients contains a continuous PVP measurement during the craniosynostosis operation while the MAC dosage is changing over time. Linear regression and MANOVA were used to test if PVP signal is influenced by MAC.

The linear regression model fit requires the input and the output to be continuous to examine the data linearity. One of the parameters to look at in linear regression is the coefficient of determination (R-squared) which measures how close the fitted line is to the data. As R-squared increases, the model shows a more linear relationship between the two continuous variables.

Rstudio was used to perform the multivariate analysis of variance (MANOVA) test. For the MANOVA test, a significance level of 0.05 was used. The Pillai's trace was the chosen test statistic due to its robustness. For the propofol waveforms, the independent variable was the classification number that was assigned to the intraoperative and preoperative PVP signals and the dependent variable was the PVP waveform. For the isoflurane waveforms, the independent variable was the MAC group, and the dependent variable was the PVP waveform.

Pairwise MANOVA was also applied for all groups of data collected from both the propofol and isoflurane data to ensure the results were reliable and are shown in Table 1.

TABLE 1
MANOVA pairwise of isoflurane patients

| | |
|---|---|
| Group 1 | [0-0.9] |
| Group 2 | [1-1.9] |
| Group 3 | [2-2.9] |
| Group 4 | [3-3.9] |

The null hypothesis in the craniosynostosis cohort patients is that as MAC dosage changes, there is no significant influence on the PVP signal. On the other hand, the alternative hypothesis states that the PVP waveform significantly changes as MAC dosage varies. For the patients whose MAC dosages were categorized into more than two MAC groups, a MANOVA pairwise test was needed to check which groups are different and which groups are the same.

The MANOVA p-values and the Pillai's trace were calculated and are shown in Tables 2 and 3 below.

TABLE 2
MANOVA results for propofol study.

| | | df | | Partial | p- |
|---|---|---|---|---|---|
| | df | error | F | h² | value |
| Hypovolemia | 50 | 327 | 6.0 | 0.478 | <0.01 |
| Euvolemia | 50 | 302 | 3.8 | 0.388 | <0.01 |

TABLE 3
MANOVA results for isoflurane study.

| Patient | | df | | Partial | p- |
|---|---|---|---|---|---|
| # | df | error | F | h² | value |
| 3 | 50 | 231 | 4.6 | 0.499 | <0.01 |
| 4 | 50 | 221 | 3.0 | 0.406 | <0.01 |
| 5 | 50 | 249 | 2.8 | 0.359 | <0.01 |
| 6 | 50 | 571 | 17.3 | 0.602 | <0.01 |
| 7 | 50 | 101 | 2.9 | 0.586 | <0.01 |
| 8 | 50 | 110 | 7.3 | 0.768 | <0.01 |
| 9 | 50 | 226 | 3.7 | 0.450 | <0.01 |

The results in the previous two tables show a significant relationship between the PVP signal and the effect of anesthetics.

### Example 5: Machine Learning Algorithms

MATLAB was used to develop k-nearest neighbor (k-NN) statistical models and build machine learning prediction systems for the propofol and isoflurane PVP waveform.

Prediction models were designed using k-nearest neighbor (k-NN) (k=1) for creating the machine learning prediction systems for the propofol and isoflurane patients. For both the propofol and isoflurane studies, 70% of the data were used for training and the remaining 30% were used for testing. However, the model parameters, /3, are unknown, and are being calculated. The training data is used to calculate the /3 coefficients and then the validation data is used to test if those calculated parameters are reliable to predict the output of the testing data correctly. Results from the machine learning systems are shown in Tables 4-9 below.

The k-nearest neighbor (k-NN) algorithm was able to classify 94 data points out of 122, 77%, for the testing data of the hypovolemic group. For the euvolemic group, the k-NN model was able to predict correctly 38 data points out of 50, 76%. Also, the algorithm was able to predict 243 data points out of 285, 85%, for the training data of the hypovolemic group and 100 data points out of 118, 85%, of the euvolemic group (Table 5). Being able to predict the class of an arbitrary PVP indicates that any volume change in the body state is detectable by the peripheral veins and machine learning can be implemented to predict the intravascular volume status of future patients without having any further information about the patient's medical record.

TABLE 4
K-NN prediction results for propofol
study out of the total number of windows.

| | Correct | Incorrect |
|---|---|---|
| | Prediction | Prediction |
| Hypovolemia | 96/106 | 10/106 |
| Euvolemia | 102/114 | 12/114 |

TABLE 5
Confusion matrix using k-nearest neighbor

| Testing data | | | Training Data | | |
|---|---|---|---|---|---|
| | Hypovol | Euvol | | Hypovol | Euvol |
| Hypovol | 94 | 28 | Hypovol | 243 | 42 |
| Euvolem | 12 | 38 | Euvolem | 18 | 100 |

The k-NN model was able to predict 78 windows out of 81, 96%, of the Preop signal for the hypovolemic group. On the other hand, the model was able to classify 20 windows out of 23, 87%, of the OR signal correctly. Also, the k-NN model was able to predict 115 out of 118, 97%, and 43 out of 54, 80%, for the training data of the Preop and OR signals, respectively (Table 6). Therefore, these results indicate that machine learning can be used to predict the volume status of future patients using only the PVP signal without the need to know the patient's medical records.

TABLE 6
Hypovolemic group confusion
matrix using k-nearest neighbor

| Testing data | | | Training Data | | |
|---|---|---|---|---|---|
| | Preop | OR | | Preop | OR |
| Preop | 78 | 3 | Preop | 115 | 3 |
| OR | 3 | 20 | OR | 11 | 43 |

The k-NN model was able to predict 96 windows out of 109, 88%, of the Preop signal for the euvolemic group. Likewise, the k-NN model predicted 37 windows correctly out of 45, 82%, of the OR signal (Table 7).

TABLE 7
Euvolemic group confusion
matrix using k-nearest neighbor

| Testing data | | | Training Data | | |
|---|---|---|---|---|---|
| | Preop | OR | | Preop | OR |
| Preop | 96 | 13 | Preop | 244 | 11 |
| OR | 8 | 37 | OR | 25 | 80 |

The correct and mismatch predictions at different isoflurane dosages for the testing and training data using k-NN are in Tables 8 and 9. The results illustrate that the change in vascular resistance is detectable in the venous circulation and the PVP signal. The machine learning system was able to accurately distinguish between the PVP waveforms of each MAC group and predict the correct MAC classification for an arbitrary PVP at least 77% of the time.

TABLE 8
K-NN prediction results for isoflurane
study out of the total number of windows.

| | Correct | Incorrect |
|---|---|---|
| Patient # | Prediction | Prediction |
| 3 | 66/82 | 16/82 |
| 4 | 55/67 | 12/67 |
| 5 | 89/90 | 1/90 |
| 6 | 143/186 | 43/186 |
| 7 | 27/35 | 8/35 |
| 8 | 23/28 | 5/28 |
| 9 | 64/82 | 18/82 |

TABLE 9
Confusion matrices of k-NN algorithm

| Patient # | Testing data | | | Training Data | | |
|---|---|---|---|---|---|---|
| 3 | | MAC 1 | MAC 2 | | MAC 1 | MAC 2 |
| | MAC 1 | 60 | 10 | MAC 1 | 164 | 0 |
| | MAC 2 | 6 | 6 | MAC 2 | 0 | 29 |
| 4 | | MAC 1 | MAC 2 | | MAC 1 | MAC 2 |
| | MAC 1 | 28 | 8 | Group 1 | 83 | 0 |
| | MAC 2 | 4 | 27 | Group 2 | 0 | 74 |
| 5 | | MAC 1 | MAC 2 | | MAC 1 | MAC 2 |
| | MAC 1 | 0 | 1 | MAC 1 | 1 | 0 |
| | MAC 2 | 0 | 89 | MAC 2 | 0 | 209 |
| 6 | | MAC 1 | MAC 2 | | MAC 1 | MAC 2 |
| | MAC 1 | 97 | 3 | MAC 1 | 234 | 0 |
| | MAC 2 | 8 | 78 | MAC 2 | 0 | 202 |
| 7 | | MAC 1 | MAC 2 | | MAC 1 | MAC 2 |
| | MAC 1 | 17 | 3 | MAC 1 | 48 | 0 |
| | MAC 2 | 5 | 10 | MAC 2 | 0 | 35 |
| 8 | | MAC 1 | MAC 2 | | MAC 1 | MAC 2 |
| | MAC 1 | 1 | 3 | MAC 1 | 10 | 0 |
| | MAC 2 | 2 | 22 | MAC 2 | 0 | 57 |
| 9 | | MAC 1 | MAC 2 | | MAC 1 | MAC 2 |
| | MAC 1 | 25 | 12 | MAC 1 | 87 | 0 |
| | MAC 2 | 6 | 39 | MAC 2 | 0 | 104 |

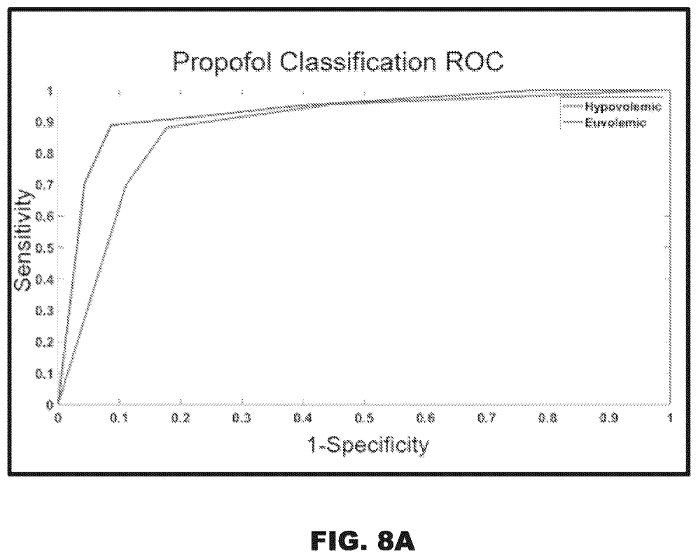

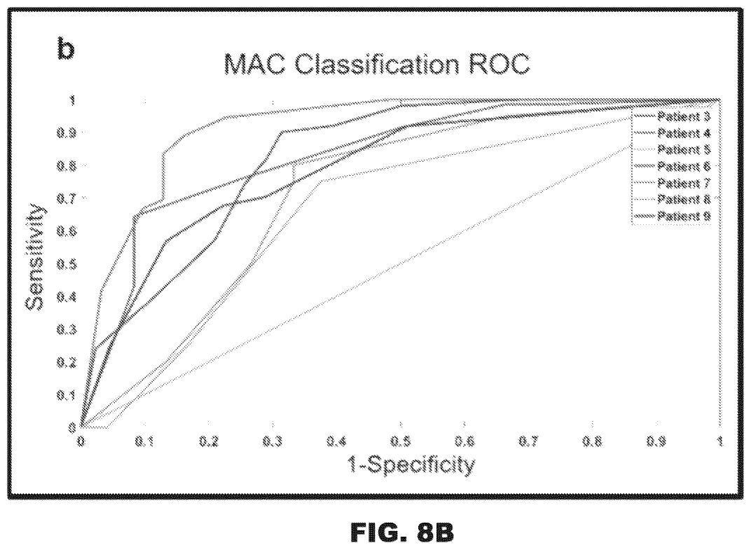

The previous tables show the number of data points that the machine learning algorithm predicted correctly for the propofol and the isoflurane. A receiver operating characteristic (ROC) curve was plotted for each cohort to illustrate the machine learning model's ability to classify the data and is shown in FIGS. 8A-8B. ROC is plotted as 1−specificity vs sensitivity, with 1−specificity=|FP|/(|FP|+|TN|) and sensitivity=|TP|/(|TP|+|FN|), where FP is false positive, FN is false negative, TP is true positive, and TN is true negative.

In addition to identifying a relationship between PVP waveforms and anesthetics, a machine learning prediction model can distinguish between PVP waveforms that have propofol and those that have no anesthetics with at least 89% accuracy, as displayed in Table 4. The ROC curve in FIG. 8A has a high area under the curve for both the hypovolemic and euvolemic data, which illustrates a high-performance measure for the machine learning model. The machine learning prediction model for the isoflurane patients accurately distinguishes between the MAC groups in each patient's PVP waveform at least 77% of the time, shown in Table 8. The ROC curve in FIG. 8B shows the highest area under the curve for patient 4, so the model has the best performance for that patient. The curves for patients 3, 6, 7, 8, and 9 show that the model is performing well at predicting the MAC groups but fails to perform for patient 5. This may be due to patient 5 having a smaller amount of clean PVP data to analyze or insufficient training data for each of the MAC groups specific to the patient. Overall, these high correct prediction results further support the conclusion that anesthetics affect the PVP waveform.

In line with the results above, even a small dosage of anesthetics can affect the PVP waveform, which captures responses to the application of anesthetics. In some examples, small dosages can be small or micro-boluses of anesthetics fluid (e.g., with a volume range from 3 to 5 cc). These small dosages of anesthetics are provided to the vein, from which the PVP waveform captures a hemodynamic state including a venous endothelium responsiveness. Such a micro-stimulus can trigger signal changes that can be captured in the waveform, which in turn helps predict, for example, fluid responsiveness or dehydration in a patient. In some examples, the responses can be monitored continuously over the course of continuous microdosing of anesthetics or other fluids.

Two additional prediction models were also tested, a logistic regression and LASSO regression model. The difference between logistic regression and LASSO regression is that the former takes all frequencies into account, even if some of them are not dominant. On the other hand, LASSO regression, which is a selection model tool, sets those unimportant parameters to zero. Therefore, the LASSO model provides a better performance with as small prediction error as possible.

The LASSO algorithm predicted correctly all the testing and training data for the hypovolemic group whereas the LASSO model did not predict correctly any data for the euvolemic group (Table 10).

TABLE 10
Confusion matrix using LASSO regression

| Testing data | | | Training Data | | |
|---|---|---|---|---|---|
| | Hypo- | Eu- | | Hypo- | Eu- |
| | volemic | volemic | | volemic | volemic |
| Hypo- | 122 | 0 | Hypo- | 285 | 0 |
| volemic | | | volemic | | |
| Eu- | 50 | 0 | Eu- | 118 | 0 |
| volemic | | | volemic | | |

The logistic regression algorithm predicted correctly 109 out of 122 for the testing data of the hypovolemic group whereas 11 were correctly predicted out of 50 for the euvolemic group. The training data was used as an input to the logistic regression system to check if the machine learning model is able to predict the data that was originally used to train the model. The algorithm predicted correctly 260 out of 285 for the training data of the hypovolemic group whereas 76 data points out of 118 were correctly predicted for the euvolemic group (Table 11).

TABLE 11
Confusion matrix using logistic regression

| Testing data | | | Training Data | | |
|---|---|---|---|---|---|
| | Hypo- | Eu- | | Hypo- | Eu- |
| | volemic | volemic | | volemic | volemic |
| Hypo- | 109 | 13 | Hypo- | 260 | 25 |
| volemic | | | volemic | | |
| Eu- | 39 | 11 | Eu- | 76 | 42 |
| volemic | | | volemic | | |

The logistic regression model was able to predict all the data points, testing and training data, correctly for the preoperative (preop) signal of the hypovolemic group. However, the prediction accuracy for the intraoperative (OR) signal for the testing and the training data was 0% (Table 12).

TABLE 12
Hypovolemic group confusion
matrix using logistic regression

| Testing data | | | Training Data | | |
|---|---|---|---|---|---|
| | Preop | OR | | Preop | OR |
| Preop | 81 | 0 | Preop | 188 | 0 |
| OR | 23 | 0 | OR | 54 | 0 |

For the testing data of the euvolemic group, the preoperative signal had 91% prediction accuracy, whereas, the intraoperative prediction accuracy was approximately 50%. For the training data, the preoperative was able to predict 250 data points correctly out of 255, 98%. Whereas 91 data points out of 105 were predicted correctly for the intraoperative signal, 87% (Table 13).

TABLE 13
Euvolemic group confusion
matrix using logistic regression

| Testing data | | | Training Data | | |
|---|---|---|---|---|---|
| | Preop | OR | | Preop | OR |
| Preop | 99 | 10 | Preop | 250 | 5 |
| OR | 21 | 24 | OR | 14 | 91 |

The LASSO regression model of the hypovolemic and euvolemic groups was able to predict correctly all the Preop data points for the training and testing data. However, the LASSO algorithm failed to predict correctly any data points of the testing and training data of the euvolemic and hypovolemic groups for the OR signal (Table 14 and 15).

TABLE 14
Hypovolemic group confusion
matrix using LASSO regression

| Testing data | | | Training Data | | |
|---|---|---|---|---|---|
| | Preop | OR | | Preop | OR |
| Preop | 81 | 0 | Preop | 188 | 0 |
| OR | 23 | 0 | OR | 54 | 0 |

TABLE 15
Euvolemic group confusion
matrix using LASSO regression

| Testing data | | | Training Data | | |
|---|---|---|---|---|---|
| | Preop | OR | | Preop | OR |
| Preop | 109 | 0 | Preop | 255 | 0 |
| OR | 45 | 0 | OR | 105 | 0 |

The linear regression and multiple logistic regression results for the craniosynostosis patients are in Tables 16 and 17. The R-squared values for all the patients are in Table 16 and the mean absolute error of linear regression was calculated and listed in Table 17.

TABLE 16
R-squared for the
linear regression of the
craniosynostosis patients

| Patient # | |
|---|---|
| 3 | 0.583 |
| 4 | 0.387 |
| 5 | 0.329 |
| 6 | 0.634 |
| 7 | 0.565 |
| 8 | 0.784 |
| 9 | 0.512 |

TABLE 17
Mean absolute error of
linear regression for the
craniosynostosis patients

| Patient | Linear |
|---|---|
| # | Regression |
| 3 | 17.38% |
| 4 | 44.33% |
| 5 | 3.09% |
| 6 | 9.06% |
| 7 | 14.88% |
| 8 | 19.06% |
| 9 | 13.58% |

The correct and mismatch predictions at different isoflurane dosages for the testing and training data using multiple logistic regression are in Table 18.

TABLE 18
Confusion matrices of craniosynostosis
using multiple logistic regression

| Patient # | Testing data | | | Training Data | | |
|---|---|---|---|---|---|---|
| 3 | | MAC 1 | MAC 2 | | MAC 1 | MAC 2 |
| | MAC 1 | 60 | 10 | MAC 1 | 163 | 1 |
| | MAC 2 | 6 | 6 | MAC 2 | 12 | 17 |
| 4 | | MAC 1 | MAC 2 | | MAC 1 | MAC 2 |
| | MAC 1 | 29 | 7 | MAC 1 | 83 | 0 |
| | MAC 2 | 13 | 18 | MAC 2 | 11 | 63 |
| 5 | | MAC 1 | MAC 2 | | MAC 1 | MAC 2 |
| | MAC 1 | 0 | 1 | MAC 1 | 1 | 0 |
| | MAC 2 | 0 | 89 | MAC 2 | 0 | 209 |
| 6 | | MAC 1 | MAC 2 | | MAC 1 | MAC 2 |
| | MAC 1 | 93 | 7 | MAC 1 | 225 | 9 |
| | MAC 2 | 8 | 78 | MAC 2 | 9 | 193 |
| 7 | | MAC 1 | MAC 2 | | MAC 1 | MAC 2 |
| | MAC 1 | 14 | 6 | MAC 1 | 48 | 0 |
| | MAC 2 | 5 | 10 | MAC 2 | 0 | 35 |
| 8 | | MAC 1 | MAC 2 | | MAC 1 | MAC 2 |
| | MAC 1 | 1 | 3 | MAC 1 | 10 | 0 |
| | MAC 2 | 3 | 21 | MAC 2 | 0 | 57 |
| 9 | | MAC 1 | MAC 2 | | MAC 1 | MAC 2 |
| | MAC 1 | 24 | 13 | MAC 1 | 74 | 13 |
| | MAC 2 | 12 | 33 | MAC 2 | 12 | 92 |

### Example 6: Dehydration and Anesthesia Influence on the Relationship Between Arterial and Venous Pressure Waveforms

The piezoelectric signal was measured along with the PVP in patients in Examples 1-5 to find if there was any correlation between the two signals. From FIGS. 2A-2B, it is clear that the two waveforms have harmonic peaks at similar frequencies. In FIG. 20, the harmonic with the highest amplitude is at 2 Hz, which is lower than the frequency, 1.2 Hz, of the highest amplitude in FIG. 2D.

The electrocardiogram (ECG/EKG) was measured along with the PVP in patients in Examples 1-5 to find if there was any correlation between the two waveforms. In FIGS. 3A-3B, the two signals have harmonic peaks at similar frequencies. In FIG. 3C, the harmonic with the highest amplitude is at 1.2 Hz, which is similar to the frequency of the highest amplitude in FIG. 3D.

In addition, data from pediatric patients was collected from 5 sequential patients undergoing surgery for pyloric stenosis. PVP and ECG waveforms were continuously collected from patients before and after the application of the anesthetic, propofol. A porcine dataset was collected on 52 healthy pigs before and after being subjected to slow bleeding. Vital signals including CVP and ECG were recorded.

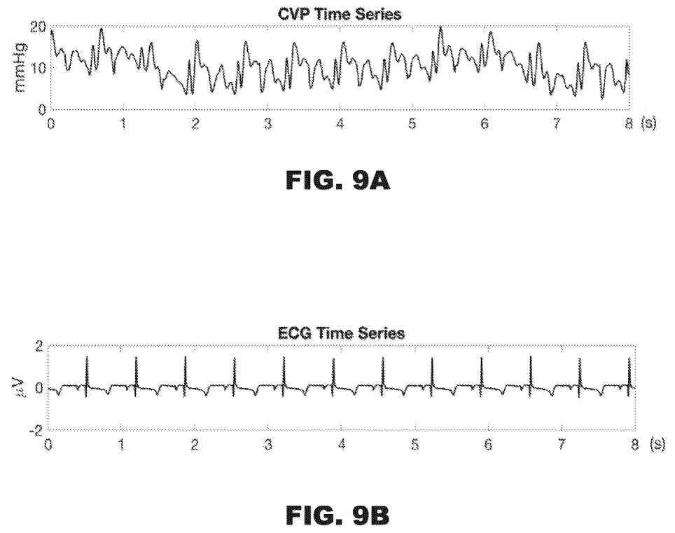

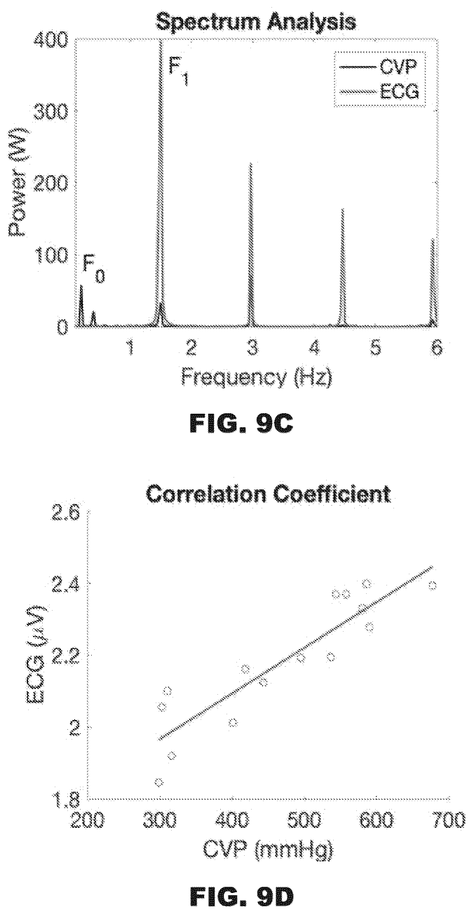

PVP and ECG waveforms were down sampled to 100 Hz and analyzed with LabChart. Motion artifacts interfering with the peripheral venous pressure waveform were removed with the pre-processing algorithm described above before signal analysis. PVP, CVP and ECG waveforms were sectioned into 2-second snippets, and an FFT was applied. A power spectral density (PSD) was plotted for each snippet and the magnitude of the amplitude of the frequencies F₀, corresponding to the respiration rate and F₁, corresponding to the pulse rate, were calculated in each snippet. A time-domain sample of a CVP and ECG waveform, along with the corresponding power spectral density with F₀ and F₁ labeled and correlation coefficient scatter plot are illustrated in FIGS. 9A-9D.

The Pearson's correlation coefficient was calculated (Eq. 1) between the PVP/CVP and ECG waveforms at the F₀ and F₁ frequencies for each subject.

$${ρ}_{X , Y} = \frac{\sum ( ( X - {μ}_{X} ) ( Y - {μ}_{Y} ) )}{{σ}_{X} {σ}_{Y}} \qquad Eq . 1$$

In the above equation, X is the magnitude of the amplitude at F₀ or F₁ from the PVP/CVP waveform and Y is the magnitude of the amplitude at F₀ or F₁ from the ECG waveform. The corresponding p-values were also recorded and a significance level of 0.05 was used.

FIG. 9A is a two second time series example of porcine CVP waveform before bleeding. FIG. 9B is a simultaneous two second time series example of the porcine ECG waveform before bleeding. FIG. 9C is a power spectral density of the CVP and ECG with respiratory rate, F₀, and pulse rate, F₁, labeled. FIG. 9D is a correlation coefficient plot at F₁. Table 19 shows all Pearson's correlation coefficients and average peak frequency (Hz) at F₀ and F₁.

TABLE 19
Pearson’s correlation coefficients

| | | Highest/ | | Highest/ |
|---|---|---|---|---|
| | Average | Lowest | Average | Lowest |
| | F₀ (Hz) | ρ at F₀ | F₁ (Hz) | ρ at F₁ |
| Animal—Before | 0.21 Hz | 0.95/0.53 | 1.51 Hz | 0.93/0.53 |
| Bleeding | | | | |
| Animal—After | 0.21 Hz | 0.94/0.54 | 1.47 Hz | 0.90/0.52 |
| Bleeding | | | | |
| Human—Before | 0.24 Hz, | 0.39/0.35 | 2.23 Hz | 0.96/0.13 |
| Anesthetic | | | | |
| Human—After | 0.25 Hz | 0.46* | 2.62 Hz | 0.96/0.57 |
| Anesthetic | | | | |

For humans before anesthetics, the average F₀ was 0.24 Hz and the average F₁ was 2.23 Hz. Only two of the five pediatric patients had a correlation coefficient at F₀ with a respective p-value below 0.05 before anesthetic application, and three had coefficients with p-values lower than 0.05 at F₁. The strongest correlation coefficient at frequency F₀ was 0.39 and the weakest 0.35. The strongest correlation coefficient at frequency F₁ was 0.96 and the weakest 0.13.

F₀ r humans after anesthetics, the average F₀ was 0.25 Hz and the average F₁ was 2.62 Hz. Only one of the five pediatric patients had a correlation coefficient at F₀ with a respective p-value below 0.05 after anesthetic application, and all five had coefficients with p-values lower than 0.05 at F₁. The coefficient at F₀ was 0.46. The strongest coefficient at F₁ was 0.96 and the weakest 0.57.

F₀ r animals before bleeding, the average F₀ was 0.21 Hz and the average F₁ was 1.51 Hz. Out of the fifty-two pigs before bleeding, 22 had correlation coefficients with a p-value below 0.05 at frequency F₁. The strongest coefficient was 0.93 and the weakest 0.53. At F₀ before bleeding, 33 pigs had coefficients with a p-value below 0.05, with the strongest being 0.95 and the weakest being 0.53.

F₀ r animals after bleeding, the average F₀ was 0.21 Hz and the average F₁ was 1.47 Hz. After bleeding, 21 of the fifty-two pigs had correlation coefficients with a p-value below 0.05 at frequency F₁. The strongest coefficient was 0.90 and the weakest 0.52. At frequency F₀, 33 pigs had coefficients with a p-value below 0.05, with the strongest being 0.94 and the weakest being 0.54.

This shows that arterial pulse pressure has a strong relationship with PVP waveforms even under the influence of strong pharmacological agents, and CVP even after large blood loss. The correlation coefficients found at F₁ using the PVP waveforms are slightly stronger than those from the CVP waveforms, which is most likely due to the difference in sampling rates between the two datasets. The pediatric dataset had a lower sampling rate of 100 Hz, resulting in an improved quality power spectral density curve for analysis.

Overall, the statistically significant correlation coefficient at F₁ is strongest in the pediatric dataset after anesthetic, which may be because of the dilation of the veins which increases proximity to nearby arteries. The strongest correlation coefficient at F₀ was present in the porcine dataset before bleeding, thus before the vessel diameters decreased due to dehydration.

In the human pediatric dataset, larger variability in the correlation coefficients at F₁ before and after the anesthetic was observed, and the coefficients at F₀ were weak in both situations. The strongest coefficient at F₁ in the pediatric dataset before anesthetic is comparable to the coefficient found after anesthetic application. Surprisingly, the correlation coefficients at F₀ and F₁ are comparable before bleeding and after bleeding in the porcine dataset, but this result does not describe before and after anesthetic, as the pigs were sedated through both stages.

Arterial pressure changes on PVP during the use of an inhaled anesthetic may be analyzed and to look at how specifically the magnitude of the amplitude at F₀ and F₁ is changing before and after anesthetic, as well as before and after mild to severe blood loss.

### Example 7: Window-Level Accuracy/Patient-Level Accuracy

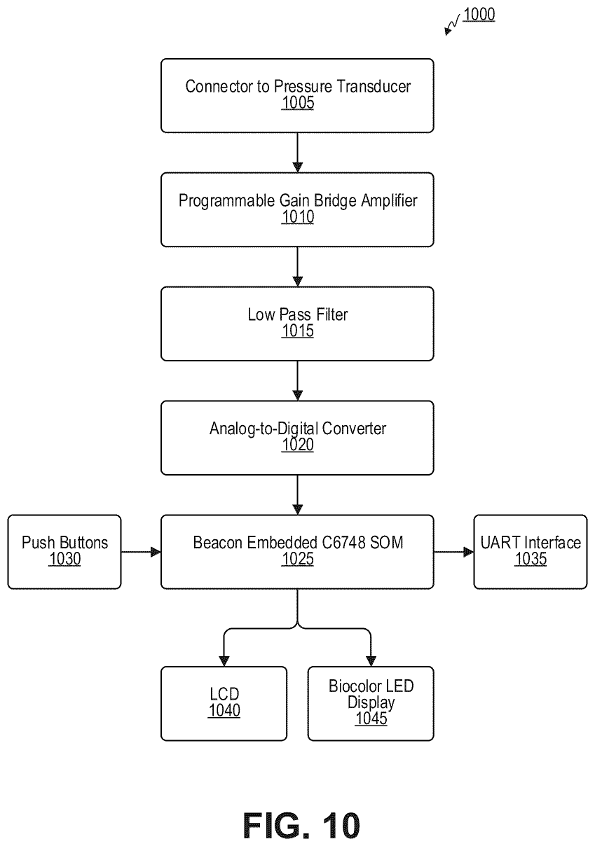

FIG. 10 is a flow diagram of an exemplary system for volume status assessment using PVP analysis. The system 1000 illustrated in this flow diagram can be an exemplary proof of concept that demonstrates the technical feasibility of a PVP acquisition and evaluation device, such as a single lumen tubular body described herein. The system 1000 comprises a single analog PVP signal channel, which processes a volume status assessment, and the system comprises a pressure transducer 1005, a programmable gain bridge amplifier 1010, a low pass filter 1015, an analog-to-digital converter 1020, and a processor 1025. In some examples, the connector to pressure transducer 1005 can be a resistive transducer that converts physical quantities into variable resistance. In some examples, the pressure transducer may comprise a resistor with 1000Ω bridge resistance, along with a ratio of change in output signal to change in input signal, the change being less than or equal to 25 μV/mmHg (i.e., sensitivity 25 μV/mmHg). In some examples, the programmable gain bridge amplifier 1010 may perform 14 gain levels, preserve waveform resolution at 2048V/V gain, measured gain of 2040V/V, 3 kHz sampling frequency, and 200 mV reference. In some examples, the low pass filter 1015 can be a higher-order butterworth filter, preserve 0 to 19.9 Hz, suppress 60 Hz noise and amplifier sampling frequency, with the measured cutoff at 29.7 Hz, and up to 35 dB/dc rolloff. In some examples, the analog-to-digital converter can preserve 14-bit resolution, 402.8 μV step size. F₀ r example, the analog-to-digital converter preserves 0.007867 mmHg step size at 2048V/V gain.

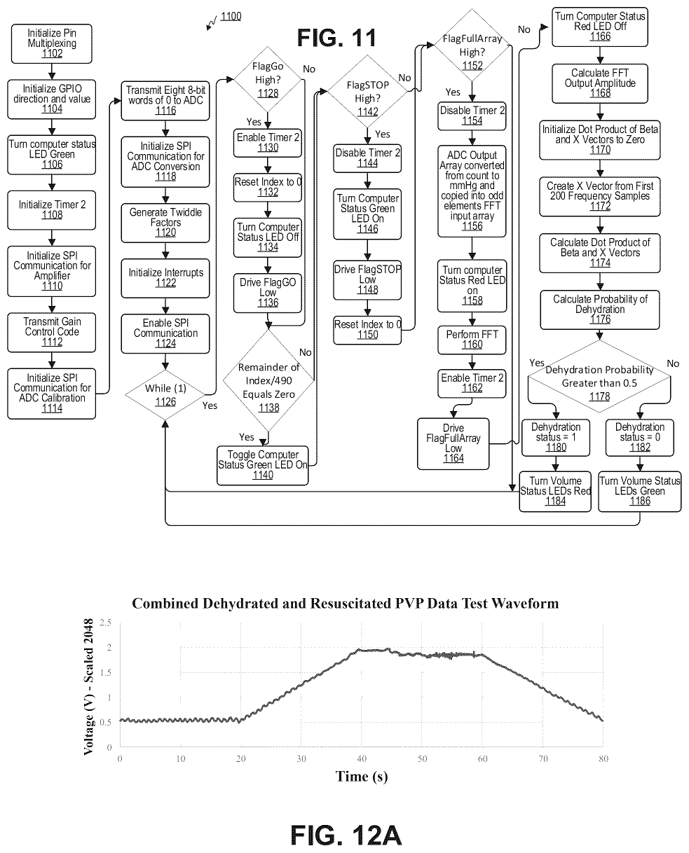

In some examples, the processor 1025 may include 128 KB shared RAM, 256 MB DDR2 RAM, 300 MHz Floating-point DSP core, DSP math library including FFT, and may apply FFT and volume prediction algorithm (the method steps of the algorithm is shows at least in FIG. 11, from step 1170 to step 1186, and corresponding descriptions of which are delineated for each of the steps). In some examples, the processor 1025 can sample PVP input at 819.2 Hz. In some examples, the volume prediction algorithm can be used to calculate probabilities of volume statuses including hydration or dehydration, anesthetic does, fluid responsiveness, sepsis, and vasoactive medication responsiveness.

In some examples, the processor 1025 may communicate with a Universal Asynchronous Receiver-Transmitter (UART) Interface 1035. In some examples, the UART interface can communicate through an external computer, a Bluetooth, a USB, or a combination thereof.

In some examples, the processor 1025 may communicate with push buttons 1030. F₀ r example, the push buttons can be a GO button or a STOP button. In some examples, the processor 1025 may be configured to an LCD panel 1040. F₀ r example, the LCD panel 1040 may be an RGB Graphic Display with the size of a display screen with 2.4 inches. In some examples, the processor 1025 may be configured to a bio-color LED Display 1045. F₀ r example, the bio-color LED display can be a red-green LED display, a computer status LED display, or two volume status LED displays.

FIG. 11 is a flow diagram of an exemplary method for volume status assessment using PVP analysis. In some examples, method can be a computer implemented method. The method comprises more than one phases (e.g., initialization phase, or sampling phase) in the volume status assessment. The first phase is an initialization phase. At step 1102, the initialization phase begins by initializing Pin Multiplexing. At step 1104, the general purpose input output (“GPIO”) direction and value are initialized. At step 1106, the computer status LED is turned green. At step 1108, timer 2 is initialized. At step 1110, SPI communication for amplifier is initialized. At step 1112, gain control code is transmitted. At step 1114, SPI communication for ADC calibration is initialized. At step 1116, Eight 8-bit words of 0 are transmitted to ADC. At step 1118, SPI communication for ADC conversion is initialized. At step 1120, Twiddle Factors are generated. At step 1122, Interrupts is initialized. At step 1124, SPI communication is enabled. At step 1126, it is determined While (1). If Yes, the method enters the sampling phase at step 1128.

The second phase is a sampling phase. At step 1128, it is determined whether [FlagGo High]. If determined Yes, the phase proceeds to step 1130. If determined No, the phase jumps to step 1138. At step 1130, Timer 2 is enabled. At step 1132, Index is reset to zero. At step 1134, computer status LED is turned off. At step 1136, FlagGo is driven low. At step 1138, it is determined whether the remainder of Index/490 equals zero. If Yes, the computer status green LED is toggled on. If No, the sampling phase is stopped at step 1142.

At step 1142, it is determined whether FlagSTOP is High or not. If Yes, Timer 2 is disabled at step 1144. If No, it is further determined whether FlagFullArray is High or not at step 1152. Once Timer 2 is disabled at step 1144, computer status green LED is turned on at step 1146. At step 1148, FlagSTOP is driven low. At step 1150, Index is reset to zero. At step 1152, it is determined whether FlagFullArray is High or not. If Yes, Timer 2 is disabled at step 1154. A new phase starts, a Frequency-Domain Pre-Processing phase, at step 1154, throughout step 1168. If it is determined No at step 1152, then the phase jumps to step 1164, where the FlagFullArray is driven low. At step 1156, an ADC output array is converted from count to mmHg and copied into odd elements FFT input array. At step 1158, the computer status red LED is turned on. At step 1160, FFT is performed. At step 1162, Timer 2 is enabled. Again at step 1164, the FlagFullArray is driven low. At step 1166, the computer status red LED is turned off. At step 1168, FFT output amplitude is calculated. The volume prediction algorithm is run beginning at step 1170 throughout 1186. In some examples, the volume prediction algorithm can calculate probabilities of various intravascular volume statuses including hydration or dehydration, anesthetics dosage, fluid responsiveness, sepsis, and vasoactive medication responsiveness. In an illustrative example, at step 1170, Dot product of Beta and X vectors are initialized to zero. At step 1172, X vector from first 200 frequency samples are created. At step 1174, Dot product of beta and X vectors are calculated. At step 1176, probability of dehydration is calculated. At step 1178, it is determined whether the calculated dehydration probability is greater than 0.5. If yes, the dehydration status is set at 1 at step 1180. If No, the dehydration status is set at 0 at step 1182. At step 1184, volume status LEDs are turned red. At step 1186, the volume status LEDs are turned green. After steps 1184 and 1186, the method returns to step 1126 for additional iterations.

MATLAB was used to develop exemplary test waveforms of dehydrated and resuscitated PVP data, combined test waveforms of the PVP data, sampled waveforms (resuscitated and dehydrated) in the time domain (x-axis) with exemplary voltage gains (e.g., 2,048 V/V and 1 V/V) applied, as well as exemplary raw waveforms (resuscitated and dehydrated) in the time domain. Additionally, a set of exemplary resuscitated PVP spectral density waveforms, illustrating a raw patient data, sampled data with 2,048 V/V gain, and 1 V/V gain, are plotted in the frequency domain (x-axis). Lastly, a set of exemplary dehydrated PVP spectral density waveforms, illustrating a raw patient data and sampled data with 1 V/V gain are plotted in the frequency domain.

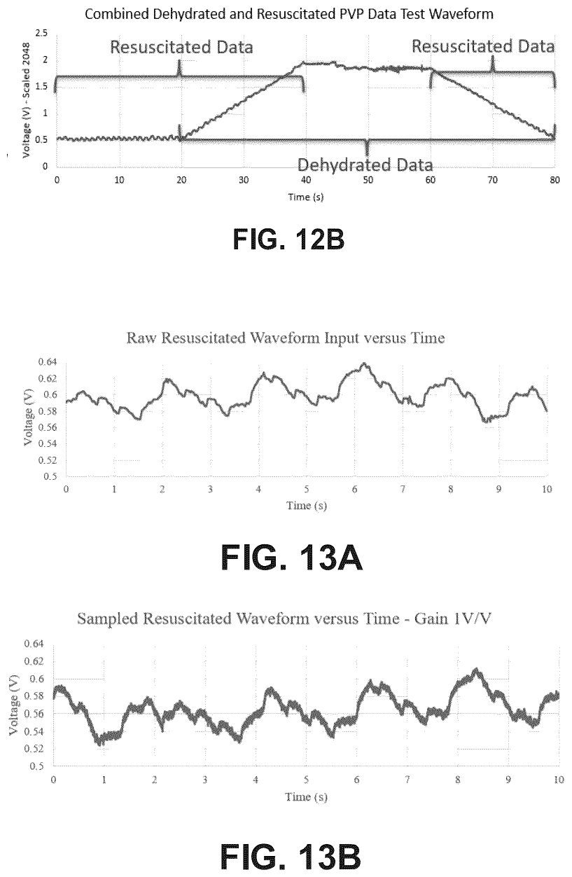

FIGS. 12A-12B show an example combined dehydrated and resuscitated PVP data test waveform in the time domain. The y-axis of the plot represents a voltage, scaled at 2,048 V/V gain. F₀ r example, the PVP amplitude in time domain represents the voltage, subject to the gain value. The x-axis of the plot represents time, with units in seconds. As illustrated in FIG. 12B, the combined PVP data test waveform can be divided in more than one segments. In some examples, the resuscitated waveform can reflect (i.e., capture) direct responses to a stimulus. In some examples, the stimulus can be fluid resuscitation or medications. In other examples, the stimulus can be a micro-stimulus such as small fluid boluses or vasoactive medication administrations. F₀ r example, a micro-bolus of 3 to 5 cc of fluid (i.e., microdosing of fluids or vasoactive medications) is provided to the vein to have the PVP waveform capture venous endothelium responsiveness. Such a micro-stimulus can trigger signal changes that can be captured in the waveform, which in turn helps predict fluid responsiveness or dehydration in a patient. In some examples, the responses can be monitored continuously over the course of a resuscitation, which also helps identify vasopressor tachyphylaxis in the vein during one or more, continuous microdosing of fluids.

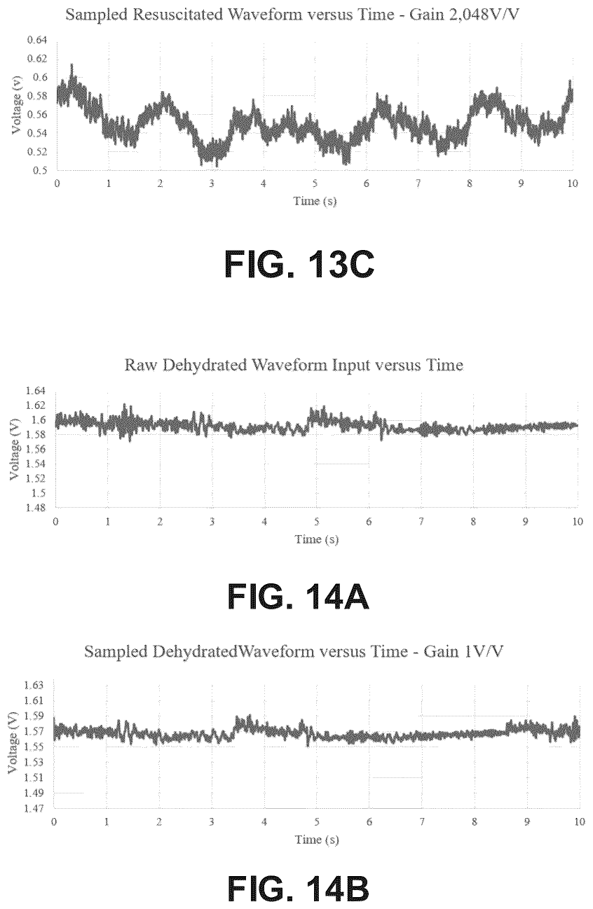

FIGS. 13A-13C show examples of raw resuscitated waveform and sampled resuscitated waveforms with exemplary voltage gains (e.g., 2,048 V/V and 1 V/V), all in the time domain. The y-axis of the plot represents a voltage. The x-axis of the plot represents time, with units in seconds.

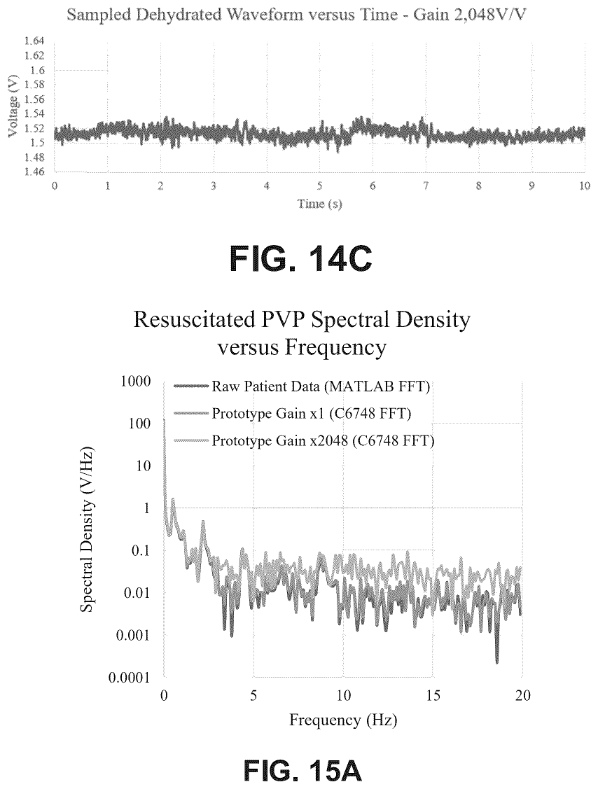

FIGS. 14A-14C show examples of raw dehydrated waveform and sampled dehydrated waveforms with exemplary voltage gains (e.g., 2,048 V/V and 1 V/V), all in the time domain. The y-axis of the plot represents a voltage. The x-axis of the plot represents time, with units in seconds.

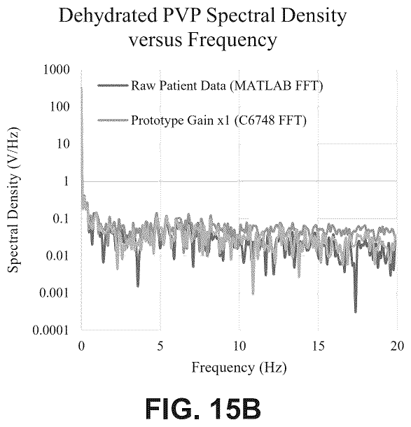

FIG. 15A shows a set of exemplary resuscitated PVP spectral density waveforms, illustrating a raw patient data, sampled data with 2,048 V/V gain, and sampled data with 1 V/V gain, plotted in the frequency domain (x-axis). The y-axis of the plot represents a spectral density, in V/Hz. The x-axis of the plot represents frequency, in Hz.

FIG. 15B shows a set of exemplary dehydrated PVP spectral density waveforms, illustrating a raw patient data and sampled data with 1 V/V gain, plotted in the frequency domain (x-axis). The y-axis of the plot represents a spectral density, in V/Hz. The x-axis of the plot represents frequency, in Hz.

Table 20 shows exemplary sets of window-level volume prediction accuracies processed in MATLAB and a prototype device, using an algorithmic volume prediction method.

TABLE 20
Window-level volume prediction accuracies

| | Algorithm Volume | Algorithm Volume |
|---|---|---|
| Clinical | Prediction | Prediction |
| Classification | Y = 0 | Y = 1 |
| | MATLAB Window-Level | |
| | Volume Prediction Accuracy | |
| Y = 0 | 93.07% (True negative, | 6.93% |
| | specificity) | (False positive) |
| Y = 1 | 2.06% | 97.94% |
| | (False negative) | (True positive, sensitivity) |
| | Prototype Window-Level | |
| | Volume Prediction Accuracy | |
| Y = 0 | 91.44% (True negative, | 6.73% |
| | specificity) | (False positive) |
| Y = 1 | 8.56% (False | 93.27% |
| | negative) | (True positive, sensitivity) |

For the prototype, the window-level volume prediction accuracy was from 1,980 windows. For MATLAB window-level volume prediction accuracy, the algorithmic volume prediction for Y=0 was 93.07%, which was true negative, and 6.93% for Y=1, which was false positive. For the prototype window-level volume prediction accuracy, the algorithmic volume prediction for Y=0 was 91.44%, which was true negative, and 6.73% for Y=1, which was false positive.

Table 21 shows exemplary sets of patient-level volume prediction accuracies processed in MATLAB and a prototype device, using an algorithmic volume prediction method.

TABLE 21
Patient-level volume prediction accuracies

| | Algorithm Volume | Algorithm Volume |
|---|---|---|
| Clinical | Prediction | Prediction |
| Classification | Y = 0 | Y= 1 |
| | MATLAB Volume Prediction Accuracy | |
| Y = 0 | 100% (True negative, | 100% (False positive) |
| | specificity) | |
| Y = 1 | 0% (False negative) | 0% (True positive, |
| | | sensitivity) |
| | Prototype Volume Prediction Accuracy | |
| Y = 0 | 97% (True negative, | 0% (False positive) |
| | specificity) | |
| Y = 1 | 3% (False negative) | 100% (True positive, |
| | | sensitivity) |

For MATLAB volume prediction accuracy, the algorithmic volume prediction for Y=0 was 100%, which was true negative, and 100% for Y=1, which was false positive. F₀ r the prototype window-level volume prediction accuracy, the algorithmic volume prediction for Y=0 was 97%, which was true negative, and 0% for Y=1, which was false positive.

Table 22 shows two sets of exemplary data, including accuracies of window-level volume status per patient and mean dehydration probability of all tested patient windows. Each set is collected by MATLAB and the prototype device.

TABLE 22
Window-Level Accuracies for resuscitated and dehydrated groups

| | | Accuracy | MATLAB | Accuracy | Prototype |
|---|---|---|---|---|---|
| | | of | Mean | of | Mean |
| | | MATLAB | Dehydration | Prototype | Dehydration |
| | | Window- | Probability | Window- | Probability |
| | | Level | of All | Level | of |
| Patient | | Volume | Tested | Volume | All Tested |
| Classi- | Patient | Status per | Patient | Status per | Patient |
| fication | Number | Patient | Windows | Patient | Windows |
| | | True | | True | |
| | | Negative | | Negative | |
| Resuscitated | 8 | 100% | 0.0072 | 100% | 0.0235 |
| Y = 0 | 9 | 100% | 0.0046 | 100% | 0.0074 |
| | 10 | 88.9% | 0.2449 | 81.1% | 0.2466 |
| | 12 | 100% | 0.0281 | 99.1% | 0.0317 |
| | 19 | 57.1% | 0.3792 | 72.9% | 0.2776 |
| | 20 | 100% | 0.0213 | 100% | 0.0020 |
| | 27 | 100% | 0.0499 | 100% | 0.0542 |
| | 28 | 66.7% | 0.2449 | 66.7% | 0.4170 |
| | 30 | 100% | 0.0058 | 100% | 0.0187 |
| | 31 | 100% | 0.0216 | 99.4% | 0.0238 |
| | | True | | True | |
| | | Positive | | Positive | |
| Dehydrated | 4 | 80% | 0.7972 | 80% | 0.7866 |
| Y = 1 | 5 | 100% | 0.9011 | 96.4% | 0.8889 |
| | 6 | 100% | 0.9936 | 100% | 0.9928 |
| | 13 | 96.3% | 0.7983 | 93.0% | 0.7712 |
| | 25 | 100% | 0.7739 | 80% | 0.6862 |
| | 26 | 100% | 0.9589 | 95.4% | 0.8796 |
| | 32 | 100% | 0.8921 | 87.5% | 0.7839 |
| | 34 | 100% | 0.9956 | 100% | 0.9840 |

For resuscitated patients, the accuracies of MATLAB window-level volume status are 100% except for patients 10, 19, and 28. The accuracies of prototype window-level volume status are also 100% except for patients 10, 12, 19, 28, and 31.

For dehydrated patients 5, 13, 25, 26, and 32, the table shows a drop in accuracy from a MATLAB algorithm to the prototype. In some examples, a sensitivity or specificity from the prototype below 95% for a given patient can be dependent on the input patient PVP data rather than error caused by the prototype system.

For patients 10, 19, and 28 from the resuscitated group, there were greater mean and standard deviation in the number of errors per test. For patients from the dehydrated group, patients 4, 13, 25, and 32 showed greater mean and standard deviation in the number of errors per test. The same patients from the respective groups also showed window-level dehydration probabilities closer to the decision level of 0.5.

As shown, eight patients showed a decrease in volume prediction accuracy from the MATLAB algorithm to the prototype. Three of these patients, patients 10, 12, and 31, were from the resuscitated group while the other five, patients 5, 13, 25, 26, and 32, were from the dehydrated group. Additionally, for all resuscitated patients except patients 19 and 20, the prototype calculated a higher probability of dehydration. For the patients from the dehydrated group, the prototype calculated a lower probability of dehydration.

Table 23 shows two sets of exemplary data, including accuracies of MATLAB volume status per patient and prototype volume status per patient.

TABLE 23
Patient-level Accuracies for resuscitated and dehydrated groups

| | | Accuracy of | Accuracy of |
|---|---|---|---|
| | | MATLAB Patient- | Prototype Patient- |
| Patient | Patient | Level Volume | Level Volume |
| Classification | Number | Status per Patient | Status per Patient |
| | | True Negative | True Negative |
| | 8 | 100% | 100% |
| | 9 | 100% | 100% |
| Resuscitated | 10 | 100% | 100% |
| Y = 0 | 12 | 100% | 100% |
| | 19 | 100% | 90% |
| | 20 | 100% | 100% |
| | 27 | 100% | 100% |
| | 28 | 100% | 80% |
| | 30 | 100% | 100% |
| | 31 | 100% | 100% |
| | | True Positive | True Positive |
| Dehydrated | 4 | 100% | 100% |
| Y = 1 | 5 | 100% | 100% |
| | 6 | 100% | 100% |
| | 13 | 100% | 100% |
| | 25 | 100% | 100% |
| | 26 | 100% | 100% |
| | 32 | 100% | 100% |
| | 34 | 100% | 100% |

As shown, patients 19 and 28 showed decreased patient-level specificity of 90% and 80%, respectively, when analyzed by the prototype. These patients also exhibited a low specificity on the window-level. The specificity of the prototype for patient 19 was 72.9%, and for patient 28, 66.7%.

Table 24 shows exemplary sets of data values associated with the prototype window-level volume status for combined patient data, with a 2,048 V/V gain.

TABLE 24
Prototype window-level volume status for combined patient data
(Gain 2,048 V/V)

| | Expected | Prototype Window-Level Volume Status for Combined | | | | | | | | | |
|---|---|---|---|---|---|---|---|---|---|---|---|
| | Volume | Patient Data | | | | | | | | | |
| | Status | (Gain 2,048 V/V) | | | | | | | | | |
| Window | per | Test | Test | Test | Test | Test | Test | Test | Test | Test | Test |
| Number | Window | 1 | 2 | 3 | 4 | 5 | 6 | 7 | 8 | 9 | 10 |
| 1 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 |
| 3 | — | 0 | 0 | 0 | 0 | 0 | 0 | 0 | 0 | 0 | 0 |
| 4 | — | 0 | 0 | 0 | 0 | 0 | 0 | 0 | 0 | 0 | 0 |
| 5 | 1 | 0 | 0 | 0 | 0 | 0 | 1 | 1 | 0 | 0 | 0 |
| 6 | 1 | 1 | 1 | 1 | 1 | 1 | 1 | 1 | 1 | 1 | 1 |
| 7 | — | 0 | 0 | 0 | 0 | 0 | 0 | 0 | 0 | 0 | 0 |
| 8 | — | 0 | 0 | 0 | 0 | 0 | 0 | 0 | 0 | 0 | 0 |

Table 25 shows exemplary sets of data values associated with the prototype window-level dehydration probability for combined patient data, with a 2,048 V/V gain.

TABLE 25
Prototype window-level dehydration probability for combined patient data
(Gain 2,048 V/V)

| | Prototype Window-Level Dehydration Probability for Combined Patient Data | | | | | | | | | |
|---|---|---|---|---|---|---|---|---|---|---|
| Window | (Gain 2,048 V/V) | | | | | | | | | |
| Number | Test 1 | Test 2 | Test 3 | Test 4 | Test 5 | Test 6 | Test 7 | Test 8 | Test 9 | Test 10 |
| 1 | 0.0075 | 0.0037 | 0.0044 | 0.0027 | 0.0011 | 0.0028 | 0.0007 | 0.0007 | 0.0022 | 0.0019 |
| 2 | 0.0039 | 0.0023 | 0.0075 | 0.0039 | 0.0029 | 0.0010 | 0.0033 | 0.0179 | 0.0016 | 0.0011 |
| 3 | 0.0007 | 0.0008 | 0.0003 | 0.0000 | 0.0001 | 0.0000 | 0.0004 | 0.0010 | 0.0007 | 0.0011 |
| 4 | 0.0011 | 0.0092 | 0.0041 | 0.0031 | 0.0086 | 0.0160 | 0.0105 | 0.0106 | 0.0029 | 0.0112 |
| 5 | 0.4143 | 0.1824 | 0.2767 | 0.1606 | 0.4107 | 0.5383 | 0.5001 | 0.1285 | 0.0763 | 0.3043 |
| 6 | 0.9880 | 0.9889 | 0.9934 | 0.9928 | 0.9942 | 0.9944 | 0.9943 | 0.9848 | 0.9956 | 0.9959 |
| 7 | 0.0004 | 0.0021 | 0.0023 | 0.0010 | 0.0023 | 0.0058 | 0.0054 | 0.0281 | 0.0002 | 0.0005 |
| 8 | 0.0005 | 0.0001 | 0.0003 | 0.0002 | 0.0000 | 0.0003 | 0.0001 | 0.0001 | 0.0004 | 0.0003 |

As shown, the dehydration probability peaked at window 6.

Having described several embodiments, it will be recognized by those skilled in the art that various modifications, alternative constructions, and equivalents may be used without departing from the spirit of the disclosure. Additionally, a number of well-known processes and elements have not been described in order to avoid unnecessarily obscuring the present disclosure. Accordingly, the above description should not be taken as limiting the scope of the disclosure.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A system for analyzing one or more conditions of a patient, the system comprising:
a device comprising:
a tubular body comprising a first lumen operable to deliver a fluid; and
at least one sensor near a tip of the tubular body, the at least one sensor configured to measure one or more characteristics of a peripheral venous pressure (PVP) waveform within a vein of the patient; and
at least one processor configured to:
receive the PVP waveform from the at least one sensor;
process the PVP waveform; and
determine a hemodynamic state of the patient based on the processed PVP waveform.

2. The system of claim 1, wherein the at least one processor is further configured to determine a change in the hemodynamic state in response to a micro-dose of fluid delivered to the patient through the first lumen.

3. The system of claim 1, wherein processing the PVP waveform includes cleaning the PVP waveform.

4. The system of claim 3, wherein cleaning the PVP waveform includes:
sectioning the PVP waveform at a pre-selected length of time to create one or more segments;
calculating a remainder of the PVP waveform divided by the pre-selected length of time;
removing any last points of the PVP waveform that are equal to the remainder;
calculating a mean and a standard deviation for each segment; and
removing a segment if there is at least one point outside a set number of standard deviations selected by a user.

5. The system of claim 2, wherein determining the hemodynamic state includes transforming the PVP waveform into a frequency domain.

6. The system of claim 1, wherein the device is configured to couple to a fluid infusion system.

7. The system of claim 1, wherein the tubular body has a second lumen, wherein the at least one sensor is disposed within the second lumen.

8. The system of claim 1, further comprising a valve in fluid communication with the first lumen.

9. The system of claim 1, wherein the device further comprises a slot in an exterior wall of the tubular body and a cover operable to enclose at least a portion of the slot, wherein the at least one sensor is disposed within the slot.

10. The system of claim 1, wherein the at least one sensor includes an absolute sensor and a barometric pressure sensor.

11. The system of claim 1, wherein the at least one sensor is operable to receive a set of calibration data.

12. A device for analyzing one or more conditions of a patient, the device comprising:
a tubular body comprising a first lumen, wherein the first lumen is operable to deliver a fluid; and
at least one sensor near a tip of the tubular body, the at least one sensor configured to measure a peripheral venous pressure within a vein of the patient.

13. The device of claim 12, wherein the tubular body comprises a second lumen, wherein the at least one sensor is disposed within the second lumen.

14. The device of claim 13, wherein the at least one sensor includes a vented pressure sensor.

15. The device of claim 12, wherein the at least one sensor is disposed within a slot at an exterior wall of the tubular body.

16. The device of claim 15, further comprising a cover, the cover operable to enclose one or more extruded wires coupled to the at least one sensor configured within the slot.

17. The device of claim 15, wherein the at least one sensor includes an absolute sensor.

18. The device of claim 17, wherein the at least one sensor further includes a barometric pressure sensor.

19. The device of claim 12, wherein the at least one sensor is operable to measure pressure at a frequency greater than a frequency of the peripheral venous pressure.

20. The device of claim 19, wherein the at least one sensor is operable to receive a set of calibration data.
